(19) United States
(12) Patent Application Publication
(10) Pub. No.: US 2020/0262930 A1
(43) Pub. Date: Aug. 20, 2020

(54) ANTI-YKL40 ANTIBODIES AND METHODS OF USE

(71) Applicant: YALE UNIVERSITY, New Haven, CT (US)

(72) Inventors: Geoffrey Chupp, Madison, CT (US); Lauren Cohn, West Hartford, CT (US)

(21) Appl. No.: 16/647,973

(22) PCT Filed: Sep. 21, 2018
(86) PCT No.: PCT/US18/52158

§ 371 Date: Mar. 17, 2020

Related U.S. Application Data
(60) Provisional application No. 62/561,258, filed on Sep. 21, 2017.

(51) Int. Cl. C07K 16/40; A61P 35/00

(52) U.S. Cl. CPC C07K 16/40; A61K 2039/505; A61P 35/00

(57) ABSTRACT

This invention relates to selective inhibition of inflammation using an anti-YKL-40 antibody. Specifically, the invention relates to methods of treating an inflammatory disease or inflammatory disorder in an individual by contacting the individual with an anti-YKL-40 antibody.

### CROSS-REFERENCE TO RELATED APPLICATION

This application claims priority to U.S. Provisional Patent Application Ser. No. 62/561,258, filed Sep. 21, 2017, which is incorporated by reference in its entirety.

### STATEMENT REGARDING FEDERALLY SPONSORED RESEARCH OR DEVELOPMENT

This invention was made with government support under NIH HL123876, NIH HL118346, NIH HL 095390, and NIH HL 095390 awarded by the National Institutes of Health. The government has certain rights in the invention.

### BACKGROUND OF THE INVENTION

Chitinase-like family of secreted proteins (CLPs) are evolutionarily conserved 18 glycosyl hydrolase proteins that bind but do not cleave chitin (Elias et al., 2005, J Allergy Clin Immunol 116:497-500). The best studied CLP is chitinase 3-like 1 (CHI3L1) that encodes the human protein YKL-40 and the mouse orthologue Brp-39. Recent studies have demonstrated that these moieties are important regulators of innate and adaptive immunity, tissue injury, apoptosis, TGF-β1 elaboration and parenchymal scarring (Rejman and Hurley, 1988, Biochem Biophys Res Commun 150:329-334; Hakala et al., 1993, J Biol Chem 268:25803-25810; Lee et al., 2009, The Journal of Experimental Medicine 206: 1149-1166; Hartl et al., 2009, Journal of Immunology 182:5098-5106). YKL-40 is produced by a variety of cells including neutrophils, monocytes, macrophages, chondrocytes, synovial cells, smooth muscle cells, endothelial and tumor cells (Johansen et al., 1995, Eur J Cancer 31A:1437-1442; Ober, and Chupp, 2009, Curr Opin Allergy Clin Immunol 9:401-408; Hakala et al., 1993, J Biol Chem 268:25803-25810) and is readily detected in the blood of normal individuals (Bojesen et al., 2011, Clin Chim Acta 412:709-712). Elevated circulating levels of YKL-40 have been observed in patients with asthma, metastatic breast cancer, cardiovascular disease, type 2 diabetes and hepatic fibrosis (Chupp et al., 2007, N Engl J Med 357:2016-2027; Shackel et al., 2003, Hepatology 38:577-588; Rathcke and Vestergaard, 2009, Cardiovasc Diabetol 8:61). In many of these disorders YKL-40 correlates with disease activity and its expression is believed to reflect distinct pathways in disease pathogenesis (Fontana et al., 2010, Gut 59:1401-1409; Thom et al., 2010, Cancer 116:4114-4121; Francescone et al., 2011, J Biol Chem. 286(17):15332-43).

Thus, there exists an unmet need in the art for a non-T2 specific inhibitor of YKL-40 for preventing and treating inflammatory disorders, such as asthma and airway remodeling.

### SUMMARY

The present invention provides an antibody that specifically binds to YKL-40. In some embodiments, the YKL-40 is human YKL-40. In some embodiments, the antibody provided by the present invention is a monoclonal antibody. In some embodiments, the antibody is a humanized antibody. In some embodiments, the antibody is a chimeric antibody. In some embodiments, the antibody comprises at least one of the vHs selected from the group consisting of: VH-V1 SEQ ID NO:1; VH-V2: SEQ ID NO:2; VH-V3: SEQ ID NO:3; VH-V4: SEQ ID NO:4; VH-V5: SEQ ID NO:5; VH-V6: SEQ ID NO: 6; VH-V7: SEQ ID NO: 7; VH-V8: SEQ ID NO: 8; VH-V9: SEQ ID NO: 9; VH-V10: SEQ ID NO: 10; VH-V11: SEQ ID NO: 11; and VH-V12: SEQ ID NO: 12. In some embodiments, the antibody comprises at least one of the vLs selected from the group consisting of: VK-V1 SEQ ID NO:13; VK-V2: SEQ ID NO:14; VK-V3: SEQ ID NO:15; VK-V4: SEQ ID NO:16; VK-V5: SEQ ID NO:17; VK-V6: SEQ ID NO:18; VK-V7: SEQ ID NO:19; and VK-V8: SEQ ID NO:20. In some embodiments, the antibody is FRG.

The present invention relates to a method of treating an inflammatory disease or disorder in an individual, comprising the step of administering to said individual the anti-YKL-40 antibody as described herein. In some embodiments, the present invention provides a method of treating a disease or disorder wherein the disease or disorder is at least selected from the group consisting of: asthma, cancer, lung cancer, lung disease, and metastatic melanoma. In some embodiments, the anti-YKL-40 antibody inhibits the activation of eosinophils.

The present invention further relates to a method of reducing the inflammation in an individual, wherein the method comprises administering an antibody to the individual, and wherein the antibody comprises a variable heavy chain region that is at least 90% identical to an amino acid sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO:11, and SEQ ID NO: 12.

In some embodiments, the present invention relates to a method of reducing the inflammation in an individual, wherein the method comprises administering an antibody to the individual, and wherein the antibody comprises a kappa variable light chain region that is at least 90% identical to an amino acid sequence selected from the group consisting of: SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20. In some embodiments, the antibody is an antibody fragment selected from the group consisting of a Fab, Fab′, F(ab)2, F(ab′)2, scFv, and combinations thereof. In some embodiments, the antibody is an antibody fragment selected from the group consisting of a Fab, Fab′, F(ab)2, F(ab′)2, scFv, and combinations thereof.

In some embodiments, the present invention relates to an antibody that specifically binds to human YKL-40, wherein the antibody has a heavy chain variable (vH) region that has an amino acid sequence that is more than 90% identical to a vH selected from the group consisting of: VH-V1 SEQ ID NO:1; VH-V2: SEQ ID NO:2; VH-V3: SEQ ID NO:3; VH-V4: SEQ ID NO:4; VH-V5: SEQ ID NO:5; VH-V6: SEQ ID NO: 6; VH-V7: SEQ ID NO: 7; VH-V8: SEQ ID NO: 8; VH-V9: SEQ ID NO: 9; VH-V10: SEQ ID NO: 10; VH-V11: SEQ ID NO: 11; and VH-V12: SEQ ID NO: 12. In some embodiments, the antibody is an antibody fragment selected from the group consisting of a Fab, Fab′, F(ab)2, F(ab′)2, scFv, and combinations thereof.

In some embodiments, the present invention relates to an antibody that specifically binds to human YKL-40, wherein the antibody has a light chain variable (vL) region that has an amino acid sequence that is more than 90% identical to an amino acid sequence selected from the group consisting of: SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20. In some embodiments, the antibody is an antibody fragment selected from the group consisting of a Fab, Fab′, F(ab)2, F(ab′)2, scFv, and combinations thereof.

In some embodiments, the present invention relates to an antibody that specifically binds to human YKL-40, wherein the antibody has a heavy chain variable (vH) region and a light chain variable (vL) region, wherein the vH region has an amino acid sequence that is more than 90% identical to one selected from the group consisting of VH-V1 SEQ ID NO:1; VH-V2: SEQ ID NO:2; VH-V3: SEQ ID NO:3; VH-V4: SEQ ID NO:4; VH-V5: SEQ ID NO:5; VH-V6: SEQ ID NO: 6; VH-V7: SEQ ID NO: 7; VH-V8: SEQ ID NO: 8; VH-V9: SEQ ID NO: 9; VH-V10: SEQ ID NO: 10; VH-V11: SEQ ID NO: 11; and VH-V12: SEQ ID NO: 12, and wherein the vL region has an amino acid sequence that is more than 90% identical to one selected from the group consisting of: SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

### BRIEF DESCRIPTION OF THE DRAWINGS

The foregoing summary, as well as the following detailed description of embodiments of the invention, will be better understood when read in conjunction with the appended drawings. It should be understood, however, that the invention is not limited to the precise arrangements and instrumentalities of the embodiments shown in the drawings. In the drawings:

FIG. 1, comprising FIG. 1A and FIG. 1B, depicts experimental results demonstrating that an anti-YKL-40 antibody blocks eosinophilic inflammation in a murine model of asthma in a dose dependent manner at <20 mg/kg every 2 weeks. *P<0.05. FIG. 1A depicts a bar graph of the quantitation of eosinophilic inflammation detected by bronchoalveolar lung lavage. FIG. 1B depicts a representative phosphor-ERK and total ERK Western blot.

FIG. 2 depicts the results of an assessment of titers of 12 stable pools and a denaturting gel showing the heavy chain and light chain of 12 stable constructs. Two constructs for each of 6 humanized variants (i.e., H1-L1, H4-L1, H11-L1, H2-L1, H11-L5, H12-L8) were generated and transfected to generate stable pools. H1-L1, H4-L1, H11-L1 (shown in bold) have the highest titers. The denaturing gel shows equal synthesis of heavy and light chains.

FIG. 3, comprising FIG. 3A and FIG. 3B, depicts results from experiments demonstrating the effects of an anti-YKL-40 antibody, FRG, on YKL-40-induced cell proliferation. Results demonstrate inhibition at both low (FIG. 3A) and high (FIG. 3B) doses of YKL-40 induced proliferation using low and high doses of FRG, respectively.

FIG. 4, comprising FIG. 4A and FIG. 4B, depicts binding of FRG to immobilized YKL-40 protein demonstrates identical binding to Kolltan-generated (FIG. 4A) and R&D generated rhYKL-40 (FIG. 4B).

FIG. 5, comprising FIG. 5A and FIG. 5B, depicts results from a titration ELISA of humanized FRG antibody against human YKL-40 (FIG. 5A) and murine BRP-30 (FIG. 5B). The 6 of the 96 clones with the best binding, species cross-reactivity, and expression characteristics are shown. hIgG1 in black is the original FRG mAb CDR grafted into a human IgG1 backbone.

FIG. 6 depicts variants 1 through 6 of the variable heavy chain (VH) of the anti-YKL-40 antibody (CDRs are underlined).

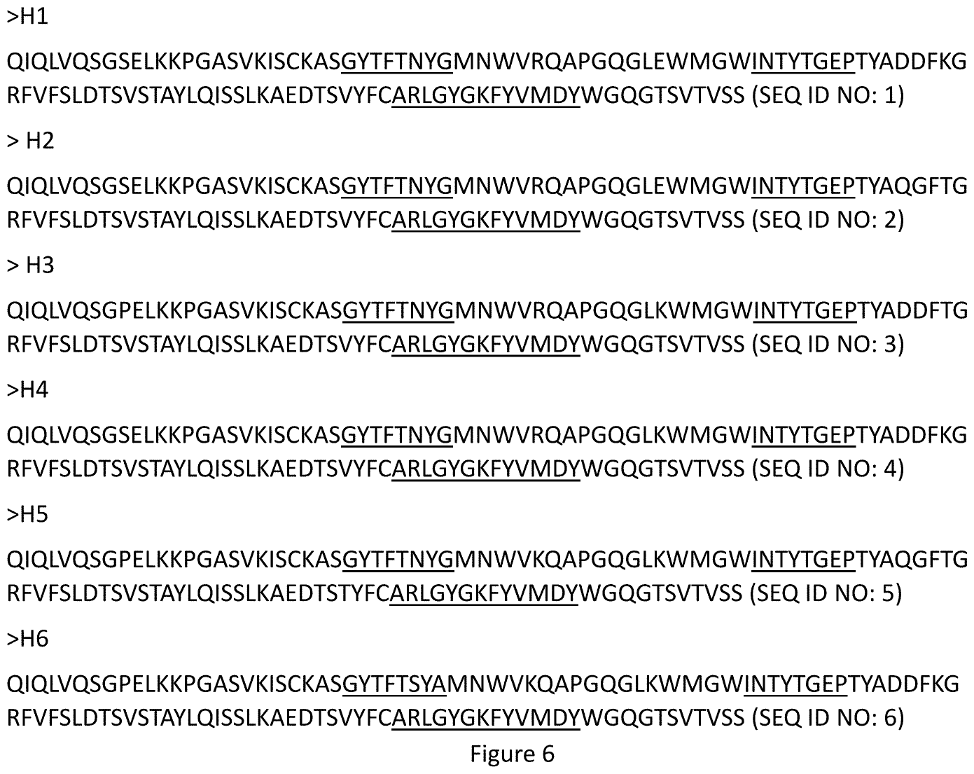

FIG. 7 depicts variants 7 through 12 of the variable heavy chain (VH) of the anti-YKL-40 antibody (CDRs are underlined).

FIG. 8 depicts variants 1 through 4 of the kappa variable light chain (VK) of the anti-YKL-40 antibody (CDRs are underlined).

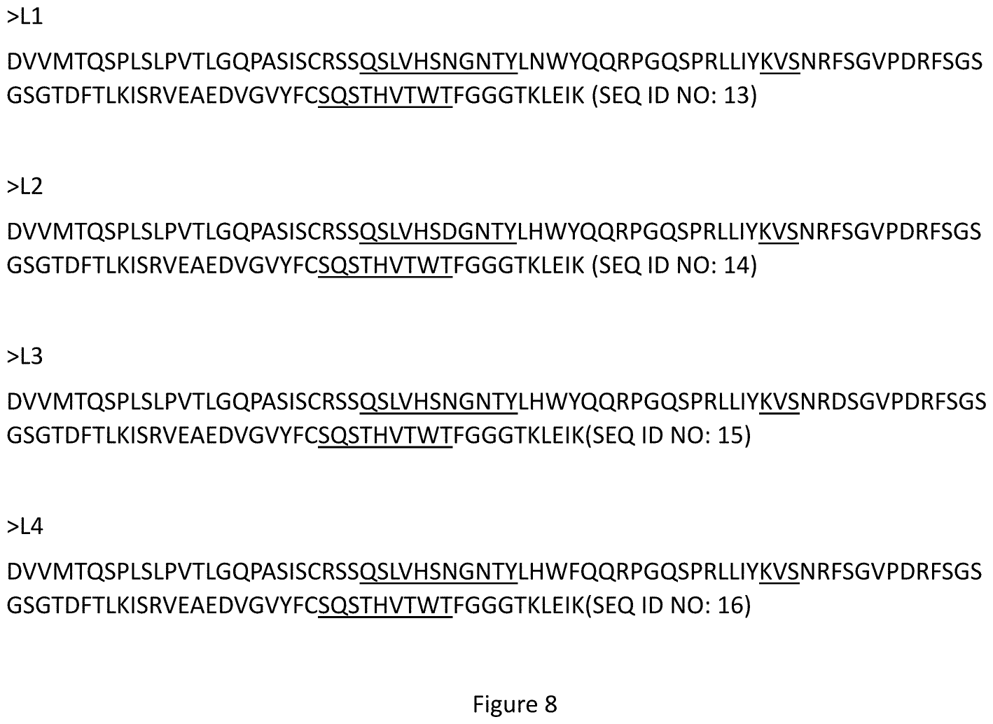

FIG. 9 depicts variants 5 through 8 of the kappa variable light chain (VK) of the anti-YKL-40 antibody (CDRs are underlined).

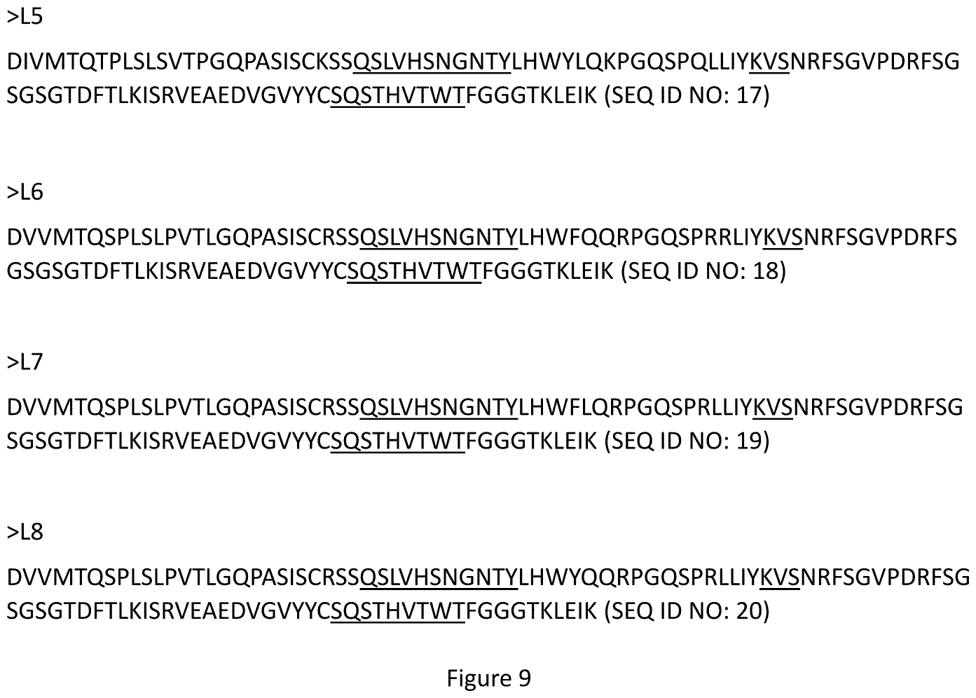

FIG. 10 depicts the results of experiments assessing the binding characteristics three stable pool antibodies ((H1L1 (green), H4L1 (grey), H11L1 (yellow)), a human/murine chimera(red), and the parental murine antibody (FRG) (blue). Results show H1L1 has superior affinity for all three species of YKL-40 or murine homologue.

FIG. 11 depicts western blots of CHI3L1/YKL40 using the anti-YKL-40 antibodies H1L1, H4L1 and FRG under denatured and non-denatured conditions.

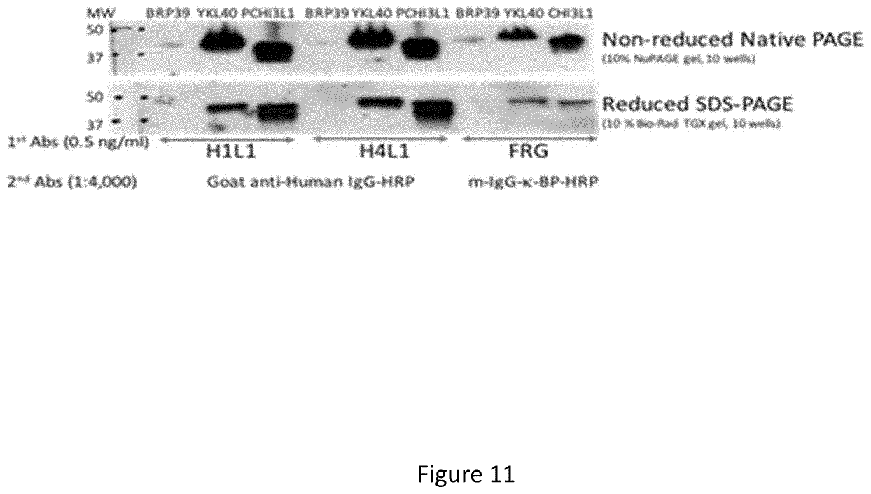

FIG. 12 depicts the results of a migration assay of human melanoma cell line comparing the blocking effects of three humanized anti-YKL-40 antibodies (i.e., H1L1, H11L1, H4L1) and FRG. H1L1 significantly blocks migration compared with the other antibodies.

FIG. 13 depicts the results of experiments assessing the inhibition of YKL-40 and IL-13 induced calcium flux by humanized anti-YKL-40 antibodies (i.e., H1L1, H4L1, H11L1). H1L1 inhibited both YKL-40 and IL-13 induced calcium flux.

FIG. 14 depicts the results of experiments assessing anti-ID scFv and IgG binding to native and denatured anti-YKL-40 H1L1. Anti-ID scFv is shown in blue and the scFv grafted into a IgG1 backbone is shown in orange. Since the scFV shows superior binding to the IgG, this reagent is being used for anti-YKL-40 PK assay. X axis is the concentration of H1L1 used for coating in the titration ELISA: 10 μg/mL, 5 μg/mL, 2.5 μg/mL, 1.25 μg/mL, 0.625 μg/mL, 0.3125 μg/mL, 0.15625 μg/mL, and 0.

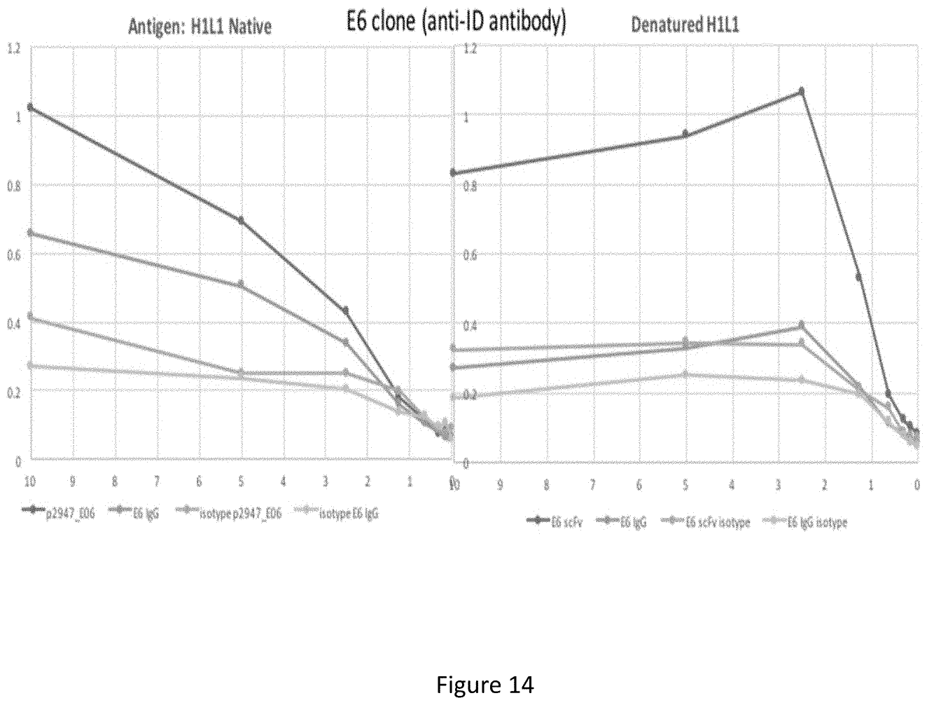

FIG. 15 depicts the results of assessments of Blood Total Protein, Albumin, Globulin and A/G Ratio in H1-L1 Treated Monkeys.

FIG. 16 depicts the results of assessments of Blood ALKP, ALT, AST and GGT in H1-L1 Treated Monkeys.

FIG. 17 depicts the results of assessments of Blood BUN, CREA, TBIL and BUN/CREA in H1L1 Treated Monkeys.

FIG. 18 depicts the results of assessments of Blood CHOL, TRIG and GLU in H1-L1 Treated Monkeys.

FIG. 19 depicts the results of assessments of Blood Ions and Na/K Ratio in H1-L1 Treated Monkeys.

FIG. 20 depicts the results of assessments of Blood Parameters in H1-L1 Treated Monkeys.

FIG. 21 depicts the results of assessments of Blood Platelet Related Parameters in H1-L1 Treated Monkeys.

FIG. 22 depicts the results of assessments of Blood Reticulocyte Related Values in H1-L1 Treated Monkeys.

FIG. 23 depicts the results of assessments of Blood Cell Differentials in H1-L1 Treated Monkeys.

FIG. 24 depicts the results of assessments of Blood Cell Differentials (%) in H1-L1 Treated Monkeys.

FIG. 25 depicts the parameters of the H1L1-treated monkey assessments.

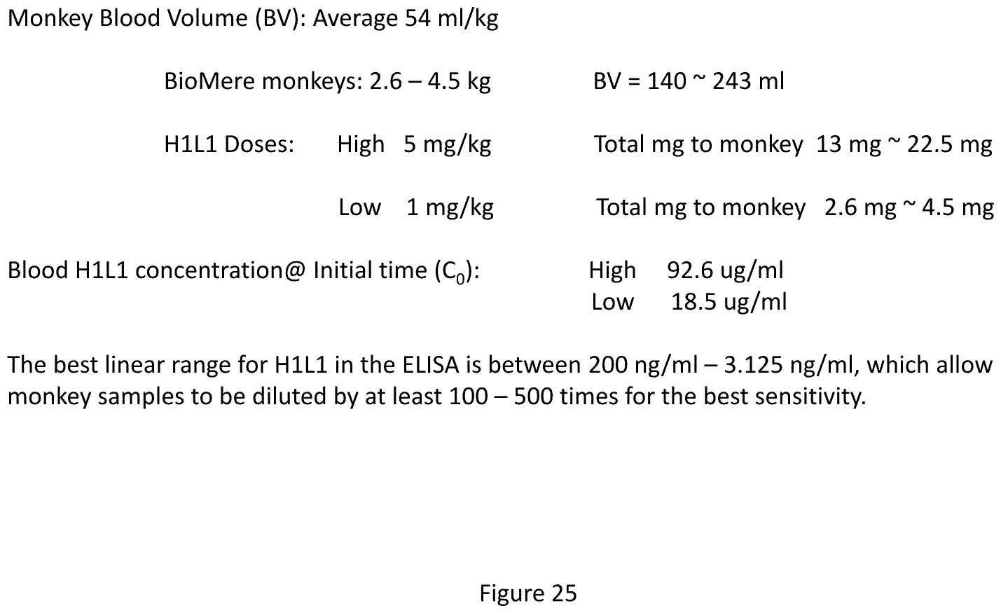

FIG. 26 depicts the results of experiments demonstrating the specific detection of H1L1/human IgG in diluted monkey serum.

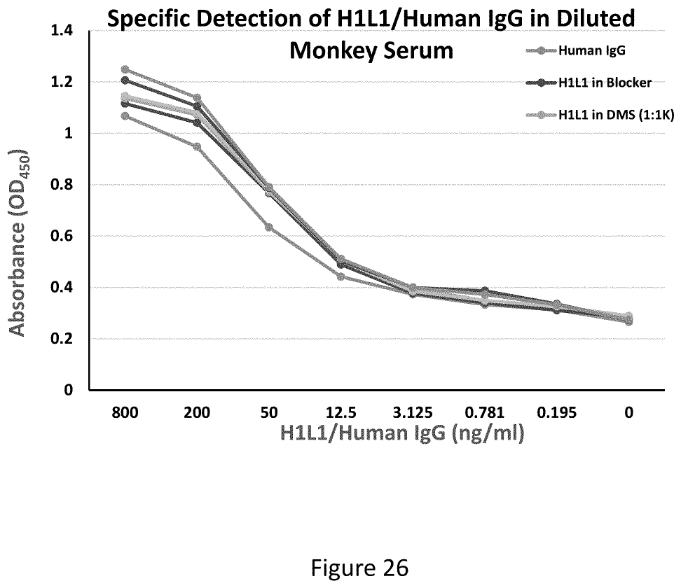

FIG. 27 depicts the results of experiments assessing the pharmacokinetics of H1L1 in monkey blood circulation.

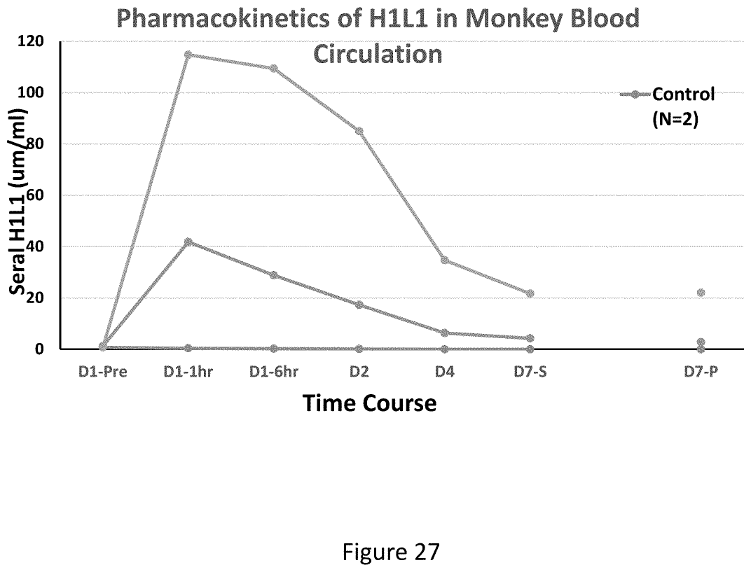

FIG. 28 depicts the results of experiments assessing the standard curve of concentration vs. mean value.

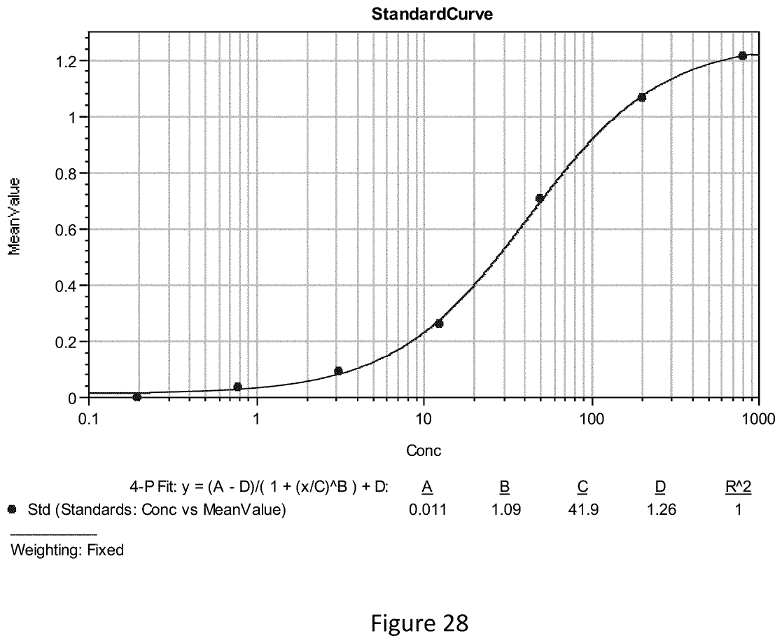

### DETAILED DESCRIPTION OF THE INVENTION

The invention relates to the inhibition of inflammatory signaling mediated by chitinases. The present invention provides compositions and methods for detecting, diagnosing, and treating inflammation in a subject. In some embodiments, the inflammation is airway inflammation. In some embodiments, the inflammation is associated with asthma. In some embodiments, the inflammation is associated with cancer. In some embodiments, the cancer is metastatic melanoma. In some embodiments, the cancer is lung cancer.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The phrase “activator,” as used herein, means to enhance a molecule, a reaction, an interaction, a gene, an mRNA, and/or a protein's expression, stability, function or activity by a measurable amount.

The term “antibody,” as used herein, refers to an immunoglobulin molecule that is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, melanoma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, pituitary, adrenal, etc.), and hematopoietic system (e.g., lymphoma, myeloma, leukemia, acute lymphocytic leukemia, chronic lymphocytic leukemia, acute myeloid leukemia, chronic myeloid leukemia, etc.).

As used herein, the term “heavy chain antibody” or “heavy chain antibodies” comprises immunoglobulin molecules derived either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

As used herein, the term “light chain antibody” or “light chain antibodies” or “light domain” or “variable light chain” comprises immunoglobulin molecules. The term light chain in mammals can refer to either lambda or kappa light chains. As used herein, light chain and kappa light chain are used interchangeably.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By the term “applicator,” as the term is used herein, is meant any device including, but not limited to, a hypodermic syringe, a pipette, and the like, for administering the compounds and compositions of the invention.

The terms “biomarker” and “marker” are used herein interchangeably. They refer to a substance that is a distinctive indicator of a biological process, biological event and/or pathologic condition.

The phrase “body sample” or “biological sample” is used herein in its broadest sense. A sample may be of any biological tissue or fluid from which biomarkers of the present invention may be assayed. Examples of such samples include but are not limited to blood, sputum, lymph, lung lavage fluids, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, sputum, saliva, sputum, tissue or fine needle biopsy samples, and archival samples with known diagnosis, treatment and/or outcome history. Biological or body samples may also include sections of tissues such as frozen sections taken for histological purposes. The sample also encompasses any material derived by processing a biological or body sample. Derived materials include, but are not limited to, cells (or their progeny) isolated from the sample, proteins or nucleic acid molecules extracted from the sample. Processing of a biological or body sample may involve one or more of: filtration, distillation, extraction, concentration, inactivation of interfering components, addition of reagents, and the like.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

In the context of the present invention, the term “control,” when used to characterize a subject, refers, by way of non-limiting examples, to a subject that is healthy, to a patient that has been diagnosed with an inflammatory disease (e.g., asthma). The term “control sample” refers to one, or more than one, sample that has been obtained from a healthy subject or from a patient diagnosed with a particular health or disease status.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold higher or more, as compared with a control.

“Differentially decreased expression” or “down regulation” refers to biomarker product levels which are at least 10% or more, for example, 20%, 30%, 40%, or 50%, 60%, 70%, 80%, 90% lower or less, and/or 0.9 fold, 0.8 fold, 0.6 fold, 0.4 fold, 0.2 fold, 0.1 fold or less, as compared with a control.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease, or disorder, the frequency with which such a sign or symptom is experienced by a patient, or both, is reduced.

Signal transduction is any process by which a cell converts one signal or stimulus into another, most often involving ordered sequences of biochemical reactions carried out within the cell. The number of proteins and molecules participating in these events increases as the process emanates from the initial stimulus resulting in a “signal cascade.” The phrase “downstream effector,” as used herein, refers to a protein or molecule acted upon during a signaling cascade, which in term acts upon another protein or molecule. The term “downstream” indicates the direction of the signaling cascade.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

The term “dysregulation” as used herein describes an over- or under-expression of chitinase 3-like 1/Brp-39/YKL-40 in an individual with inflammation or inflammatory disease as compared to a normal individual without inflammation or inflammatory disease.

As used herein “endogenous” refers to any material from or produced inside the organism, cell, tissue or system.

As used herein, the term “exogenous” refers to any material introduced from or produced outside the organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The “level” of one or more biomarkers means the absolute or relative amount or concentration of the biomarker in the sample.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “normal” and “healthy” are used herein interchangeably. They include an individual or group of individuals who have not been diagnoses with or have symptoms of asthma, cancer, or any other relevant pathology and who have not shown any signs or symptoms of injury, damage or dysfunction associated with inflammation or otherwise. The term “normal” is also used herein to qualify a sample (e.g., a blood sample) obtained from a healthy individual.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

By the term “specifically binds,” as used herein, is meant a molecule, such as an antibody, which recognizes and binds to another molecule or feature, but does not substantially recognize or bind other molecules or features in a sample.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “subject” refers to a human or another mammal (e.g., primate, dog, cat, goat, horse, pig, mouse, rat, rabbit, and the like). In many embodiments of the present invention, the subject is a human being. In such embodiments, the subject is often referred to as an “individual” or a “patient.” The terms “individual” and “patient” do not denote a particular age.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based on the discovery of the role of chitinase 3-like 1/Brp-39/YKL-40 (i.e., YKL-40) in inflammation and inflammatory diseases and disorders such as asthma and cancer, its roles in the pathogenesis of cancer and lung diseases and disorders, such as asthma, and its utility as a biomarker for inflammatory diseases and disorders, such as asthma and cancer. The invention is also based on the unexpected discovery of the role of chitinase-like proteins in lung inflammation and the concept that the innate immune response has evolved to identify and dispose of cells that are severely injured while promoting the survival and expansion of sublethally injured cells to affect subsequent organ repair.

The discovery of chitinase 3-like 1/Brp-39/YKL-40 as both a sensor of the degree of inflammation and a critical mediator of the reparative response in inflammatory diseases such as those of the lung provides a powerful biomarker that can be used to rapidly identify patients that are at greater risk of having lung inflammation, including lung inflammation associated with asthma and/or cancer. Accordingly, the invention provides compositions and methods useful in identifying an individual with an inflammatory disease or disorder, or an individual at risk of developing an inflammatory disease or disorder. In one aspect of the invention, the individual is a mammal. In another aspect of the invention, the mammal is a human.

This invention relates to the inhibition of inflammatory signaling mediated by chitinases. The present invention provides compositions and methods for detecting, diagnosing, and treating inflammation in a subject. In some embodiments, the inflammation is airway inflammation. In some embodiments, the inflammation is associated with asthma. In some embodiments, the inflammation is associated with cancer. In some embodiments, the cancer is metastatic cancer, such as metastatic melanoma. In some embodiments, the cancer is lung cancer.

In some embodiments, the invention is a method of inhibiting inflammation induced by YKL-40 in an individual, comprising the step of administering to said individual at least one anti-YKL-40 antibody, and thereby inhibiting YKL-40 activation in an individual.

### Anti-YKL-40 Antibodies

In some embodiments, the invention includes compositions comprising at least one antibody that specifically binds to YKL-40. In one embodiment, the anti-YKL-40 antibody is a polyclonal antibody. In another embodiment, the anti-YKL-40 antibody is a monoclonal antibody. In some embodiments, the anti-YKL-40 antibody is a chimeric antibody. In further embodiments, the anti-YKL-40 antibody is a humanized antibody. In some embodiments, the antibody is an antibody fragment. In some embodiments, the YKL-40 is human YKL-40.

In some embodiments, the invention is a protein or a polypeptide capable of binding to human YKL-40. In some embodiments, the antibody or antibody fragment; the protein or the polypeptide binds to a relevant portion or fraction or epitope of the human-YKL-40; and the binding of the antibody, or the antibody fragment thereof, or the protein or the polypeptide to the relevant portion of the human-YKL-40 is associated with a reduction in inflammation in an intact organism.

In some embodiments, the peptide that binds to the relevant portion of the human-YKL-40, is a cyclized peptide. In some embodiments, the peptide that binds to the relevant portion of the human-YKL-40 is a modified peptide. In some cases, the human-YKL-40 binding antibody or a YKL-40 binding antibody fragment thereof, is further conjugated to a protein, a peptide or another compound.

In one embodiment, the anti-YKL-40 antibody or an antigen-binding fragment thereof comprises a heavy chain variable (vH) region selected from the group consisting of: VH-V1 SEQ ID NO:1 (i.e., H1); VH-V2: SEQ ID NO:2 (i.e., H2); VH-V3: SEQ ID NO:3 (i.e., H3); VH-V4: SEQ ID NO:4 (i.e., H4); VH-V5: SEQ ID NO:5 (i.e., H5); VH-V6: SEQ ID NO: 6 (i.e., H6); VH-V7: SEQ ID NO: 7 (i.e., H7); VH-V8: SEQ ID NO: 8 (i.e., H8); VH-V9: SEQ ID NO: 9 (i.e., H9); VH-V10: SEQ ID NO: 10 (i.e., H10); VH-V11: SEQ ID NO: 11 (i.e., H11); and VH-V12: SEQ ID NO: 12 (i.e., H12). In one embodiment, the anti-YKL-40 antibody or an antigen-binding fragment thereof comprises a heavy chain variable (vH) region having a sequence that is at least about 85% (such as at least about any of 85%, 86%, 87%, 88%, 89%, 90%, 91%, 92%, 93%, 94%, 95%, 96%, 97%, 98%, 99%, or 100%) identical to VH-V1 SEQ ID NO:1 (i.e., H1); VH-V2: SEQ ID NO:2 (i.e., H2); VH-V3: SEQ ID NO:3 (i.e., H3); VH-V4: SEQ ID NO:4 (i.e., H4); VH-V5: SEQ ID NO:5 (i.e., H5); VH-V6: SEQ ID NO: 6 (i.e., H6); VH-V7: SEQ ID NO: 7 (i.e., H7); VH-V8: SEQ ID NO: 8 (i.e., H8); VH-V9: SEQ ID NO: 9 (i.e., H9); VH-V10: SEQ ID NO: 10 (i.e., H10); VH-V11: SEQ ID NO: 11 (i.e., H11); and VH-V12: SEQ ID NO: 12 (i.e., H12).

In one embodiment, the anti-YKL-40 antibody or an antigen-binding fragment thereof comprises a kappa light chain variable (vK or vL) region selected from the group consisting of: VK-V1 SEQ ID NO:13 (i.e., L1); VK-V2: SEQ ID NO:14 (i.e., L2); VK-V3: SEQ ID NO:15 (i.e., L3); VK-V4: SEQ ID NO:16 (i.e., L4); VK-V5: SEQ ID NO:17 (i.e., L5); VK-V6: SEQ ID NO:18 (i.e., L6); VK-V7: SEQ ID NO:19 (i.e., L7); and VK-V8: SEQ ID NO:20 (i.e., L8). In one embodiment, the anti-YKL-40 antibody or an antigen-binding fragment thereof comprises a kappa light chain variable (vK or vL) region having a sequence that is at least about 85% (such as at least about any of 85%, 86%, 87%, 88%, 89%, 90%, 91%, 92%, 93%, 94%, 95%, 96%, 97%, 98%, 99%, or 100%) identical to VK-V1 SEQ ID NO:13; VK-V2: SEQ ID NO:14; VK-V3: SEQ ID NO:15; VK-V4: SEQ ID NO:16; VK-V5: SEQ ID NO:17; VK-V6: SEQ ID NO:18; VK-V7: SEQ ID NO:19; or VK-V8: SEQ ID NO:20.

In some embodiments, the anti-YKL-40 antibody or an antigen binding fragment thereof comprises a heavy chain variable region comprising the amino acid sequence of at least one of SEQ ID NO:1 through SEQ ID NO: 12. In other embodiments, the anti-YKL-40 antibody comprises a kappa light chain variable region comprising at least one of the amino acid sequences of SEQ ID NO:13 through SEQ ID NO: 20. In some embodiments, the anti-YKL-40 antibody is humanized. In some embodiments, the anti-YKL-40 antibody is a chimeric antibody.

In one embodiment, the anti-YKL-40 antibody comprises a vH that is SEQ ID NO:1 and a vL that is SEQ ID NO:13 (i.e., H1-L1). In one embodiment, the anti-YKL-40 antibody comprises a vH that is SEQ ID NO:4 and a vL that is SEQ ID NO:13 (i.e., H4-L1). In one embodiment, the anti-YKL-40 antibody comprises a vH that is SEQ ID NO:11 and a vL that is SEQ ID NO:13 (i.e., H11-L1). In one embodiment, the anti-YKL-40 antibody comprises a vH that is SEQ ID NO:2 and a vL that is SEQ ID NO:13 (i.e., H2-L1). In one embodiment, the anti-YKL-40 antibody comprises a vH that is SEQ ID NO:11 and a vL that is SEQ ID NO:17 (i.e., H11-L5). In one embodiment, the anti-YKL-40 antibody comprises a vH that is SEQ ID NO:12 and a vL that is SEQ ID NO:20 (i.e., H12-L8).

In some embodiments, the antibodies are chimeric antibodies. In some embodiments, the anti-human YKL-40 antibody comprises a combination of human kappa light chain and human heavy chain regions sequences described elsewhere in the specification. One of skill in the art would be able to prepare and obtain a chimeric antibody using known techniques of swapping relevant domains of specific antibodies of interest. Such an antibody is easily prepared by grafting heterogeneous antibody domains, incorporating one or more sequences described in this application. Using known recombinant technology, it is possible to obtain and prepare a recombinant antibody comprising heavy and light chain constant regions encoded by nucleic acid sequences of human heavy and light chain constant regions; and the heavy and light chain variable regions encoded by nucleic acid sequences corresponding to the sequences set forth in the disclosure. One of skill in the art can prepare an anti-human YKL-40 antibody comprises one or more vH or vL sequences described in this disclosure, wherein portions of the light chain alone or portions of the heavy chain alone are replaced with regions from an antibody belonging to another species, for example human. A human anti-human-YKL-40 antibody comprising variable regions having one or more vH or vL sequence selected from SEQ ID NOs: 1-20, in combination with murine or non-murine antibody structural elements can be prepared by routine methods known in the art. In some embodiments, the antibodies or antibody fragments are further humanized using known techniques in the art.

In some embodiments, the anti-YKL-40 antibody comprises an antibody having at least about 80% (such as at least about any of 80%, 81%, 82%, 83%, 84%, 85%, 86%, 87%, 88%, 89%, 90%, 91%, 92%, 93%, 94%, 95%, 96%, 97%, 98%, 99%, or 100%) amino acid identity with the vH or vL sequences described herein, listed in SEQ ID NOs 1-20.

In one embodiment, the anti-YKL-40 antibody or an antigen-binding fragment thereof comprises at least one of the CDRs selected from the group consisting of: VH-CDR1: SEQ ID NO:21 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof. In another embodiment, the anti-YKL-40 antibody comprises all of the CDRs of the group consisting of: VH-CDR1: SEQ ID NO:21 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof.

In some embodiments, the anti-YKL-40 antibody or antigen-binding fragment thereof comprises: VH-CDR1 comprising the amino acid sequence of SEQ ID NO:21 or a variant thereof comprising up to about 3 (such as about any of 1, 2, or 3) amino acid substitutions; and VL-CDR1: SEQ ID NO:26, or a variant thereof comprising up to about 3 (such as about any of 1, 2, or 3) amino acid substitutions. In some embodiments, the anti-YKL-40 antibody or an antigen-binding fragment thereof comprises: VH-CDR1 comprising the amino acid sequence of SEQ ID NO:21; and VL-CDR1: SEQ ID NO:26.

In some embodiments, the anti-YKL-40 antibody or antigen-binding fragment thereof comprises: VH-CDR2 comprising the amino acid sequence of SEQ ID NO:22, or a variant thereof comprising up to about 3 (such as about any of 1, 2, or 3) amino acid substitutions; and VL-CDR2: SEQ ID NO:27, or a variant thereof comprising up to about 3 (such as about any of 1, 2, or 3) amino acid substitutions. In some embodiments, the anti-YKL-40 antibody or an antigen-binding fragment thereof comprises: VH-CDR2 comprising the amino acid sequence of SEQ ID NO:22; and VL-CDR2: SEQ ID NO:27.

In some embodiments, the anti-YKL-40 antibody or antigen-binding fragment thereof comprises: VH-CDR3 comprising the amino acid sequence of SEQ ID NO:23, or a variant thereof comprising up to about 3 (such as about any of 1, 2, or 3) amino acid substitutions; and VL-CDR3: SEQ ID NO:28, or a variant thereof comprising up to about 3 (such as about any of 1, 2, or 3) amino acid substitutions. In some embodiments, the anti-YKL-40 antibody or an antigen-binding fragment thereof comprises: VH-CDR3 comprising the amino acid sequence of SEQ ID NO:23; and VL-CDR3: SEQ ID NO:28.

In some embodiments, the anti-YKL-40 antibody or antigen-binding fragment thereof comprises: VH-CDR1 comprising the amino acid sequence of SEQ ID NO:21, or a variant thereof comprising up to about 3 (such as about any of 1, 2, or 3) amino acid substitutions; VH-CDR2 comprising the amino acid sequence of SEQ ID NO:22, or a variant thereof comprising up to about 3 (such as about any of 1, 2, or 3) amino acid substitutions; VH-CDR3 comprising the amino acid sequence of SEQ ID NO:23, or a variant thereof comprising up to about 3 (such as about any of 1, 2, or 3) amino acid substitutions; VL-CDR1 comprising the amino acid sequence of SEQ ID NO:26, or a variant thereof comprising up to about 3 (such as about any of 1, 2, or 3) amino acid substitutions; VL-CDR2 comprising the amino acid sequence of SEQ ID NO:27, or a variant thereof comprising up to about 3 (such as about any of 1, 2, or 3) amino acid substitutions; and VL-CDR3 comprising the amino acid sequence of SEQ ID NO:28, or a variant thereof comprising up to about 3 (such as about any of 1, 2, or 3) amino acid substitutions.

In one embodiment, the anti-YKL-40 antibody or an antigen-binding fragment thereof comprises at least one of the CDRs selected from the group consisting of: VH-CDR1: SEQ ID NO:24 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof. In another embodiment, the anti-YKL-40 antibody comprises all of the CDRs of the group consisting of: VH-CDR1: SEQ ID NO:24 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof.

In some embodiments, the anti-YKL-40 antibody or antigen-binding fragment thereof comprises: VH-CDR1 comprising the amino acid sequence of SEQ ID NO:24 or a variant thereof comprising up to about 3 (such as about any of 1, 2, or 3) amino acid substitutions; and VL-CDR1: SEQ ID NO:26, or a variant thereof comprising up to about 3 (such as about any of 1, 2, or 3) amino acid substitutions. In some embodiments, the anti-YKL-40 antibody or an antigen-binding fragment thereof comprises: VH-CDR1 comprising the amino acid sequence of SEQ ID NO:24; and VL-CDR1: SEQ ID NO:26.

In some embodiments, the anti-YKL-40 antibody or antigen-binding fragment thereof comprises: VH-CDR2 comprising the amino acid sequence of SEQ ID NO:22, or a variant thereof comprising up to about 3 (such as about any of 1, 2, or 3) amino acid substitutions; and VL-CDR2: SEQ ID NO:27, or a variant thereof comprising up to about 3 (such as about any of 1, 2, or 3) amino acid substitutions. In some embodiments, the anti-YKL-40 antibody or an antigen-binding fragment thereof comprises: VH-CDR2 comprising the amino acid sequence of SEQ ID NO:22; and VL-CDR2: SEQ ID NO:27.

In some embodiments, the anti-YKL-40 antibody or antigen-binding fragment thereof comprises: VH-CDR3 comprising the amino acid sequence of SEQ ID NO:23, or a variant thereof comprising up to about 3 (such as about any of 1, 2, or 3) amino acid substitutions; and VL-CDR3: SEQ ID NO:28, or a variant thereof comprising up to about 3 (such as about any of 1, 2, or 3) amino acid substitutions. In some embodiments, the anti-YKL-40 antibody or an antigen-binding fragment thereof comprises: VH-CDR3 comprising the amino acid sequence of SEQ ID NO:23; and VL-CDR3: SEQ ID NO:28.

In some embodiments, the anti-YKL-40 antibody or antigen-binding fragment thereof comprises: VH-CDR1 comprising the amino acid sequence of SEQ ID NO:24, or a variant thereof comprising up to about 3 (such as about any of 1, 2, or 3) amino acid substitutions; VH-CDR2 comprising the amino acid sequence of SEQ ID NO:21, or a variant thereof comprising up to about 3 (such as about any of 1, 2, or 3) amino acid substitutions; VH-CDR3 comprising the amino acid sequence of SEQ ID NO:23, or a variant thereof comprising up to about 3 (such as about any of 1, 2, or 3) amino acid substitutions; VL-CDR1 comprising the amino acid sequence of SEQ ID NO:26, or a variant thereof comprising up to about 3 (such as about any of 1, 2, or 3) amino acid substitutions; VL-CDR2 comprising the amino acid sequence of SEQ ID NO:27, or a variant thereof comprising up to about 3 (such as about any of 1, 2, or 3) amino acid substitutions; and VL-CDR3 comprising the amino acid sequence of SEQ ID NO:28, or a variant thereof comprising up to about 3 (such as about any of 1, 2, or 3) amino acid substitutions.

In one embodiment, the anti-YKL-40 antibody or an antigen-binding fragment thereof comprises at least one of the CDRs selected from the group consisting of: VH-CDR1: SEQ ID NO:25 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof. In another embodiment, the anti-YKL-40 antibody comprises all of the CDRs of the group consisting of: VH-CDR1: SEQ ID NO:25 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof.

In some embodiments, the anti-YKL-40 antibody or antigen-binding fragment thereof comprises: VH-CDR1 comprising the amino acid sequence of SEQ ID NO:25 or a variant thereof comprising up to about 3 (such as about any of 1, 2, or 3) amino acid substitutions; and VL-CDR1: SEQ ID NO:26, or a variant thereof comprising up to about 3 (such as about any of 1, 2, or 3) amino acid substitutions. In some embodiments, the anti-YKL-40 antibody or an antigen-binding fragment thereof comprises: VH-CDR1 comprising the amino acid sequence of SEQ ID NO:25; and VL-CDR1: SEQ ID NO:26.

In some embodiments, the anti-YKL-40 antibody or antigen-binding fragment thereof comprises: VH-CDR2 comprising the amino acid sequence of SEQ ID NO:22, or a variant thereof comprising up to about 3 (such as about any of 1, 2, or 3) amino acid substitutions; and VL-CDR2: SEQ ID NO:27, or a variant thereof comprising up to about 3 (such as about any of 1, 2, or 3) amino acid substitutions. In some embodiments, the anti-YKL-40 antibody or an antigen-binding fragment thereof comprises: VH-CDR2 comprising the amino acid sequence of SEQ ID NO:22; and VL-CDR2: SEQ ID NO:27.

In some embodiments, the anti-YKL-40 antibody or antigen-binding fragment thereof comprises: VH-CDR3 comprising the amino acid sequence of SEQ ID NO:23, or a variant thereof comprising up to about 3 (such as about any of 1, 2, or 3) amino acid substitutions; and VL-CDR3: SEQ ID NO:28, or a variant thereof comprising up to about 3 (such as about any of 1, 2, or 3) amino acid substitutions. In some embodiments, the anti-YKL-40 antibody or an antigen-binding fragment thereof comprises: VH-CDR3 comprising the amino acid sequence of SEQ ID NO:23; and VL-CDR3: SEQ ID NO:28.

In some embodiments, the anti-YKL-40 antibody or antigen-binding fragment thereof comprises: VH-CDR1 comprising the amino acid sequence of SEQ ID NO:25, or a variant thereof comprising up to about 3 (such as about any of 1, 2, or 3) amino acid substitutions; VH-CDR2 comprising the amino acid sequence of SEQ ID NO:22, or a variant thereof comprising up to about 3 (such as about any of 1, 2, or 3) amino acid substitutions; VH-CDR3 comprising the amino acid sequence of SEQ ID NO:23, or a variant thereof comprising up to about 3 (such as about any of 1, 2, or 3) amino acid substitutions; VL-CDR1 comprising the amino acid sequence of SEQ ID NO:26, or a variant thereof comprising up to about 3 (such as about any of 1, 2, or 3) amino acid substitutions; VL-CDR2 comprising the amino acid sequence of SEQ ID NO:27, or a variant thereof comprising up to about 3 (such as about any of 1, 2, or 3) amino acid substitutions; and VL-CDR3 comprising the amino acid sequence of SEQ ID NO:28, or a variant thereof comprising up to about 3 (such as about any of 1, 2, or 3) amino acid substitutions.

In one embodiment, the anti-YKL-40 antibody or an antigen-binding fragment thereof comprises at least one of the CDRs selected from the group consisting of: VH-CDR1: SEQ ID NO:21 VH-CDR2: SEQ ID NO:22; VH-CDR3: SEQ ID NO:23; VL-CDR1: SEQ ID NO:29; VL-CDR2: SEQ ID NO:27; and VL-CDR3: SEQ ID NO:28, or a variant or variants thereof. In another embodiment, the anti-YKL-40 antibody comprises all of the CDRs of the group consisting of: VH-CDR1: SEQ ID NO:21 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof.

In some embodiments, the anti-YKL-40 antibody or antigen-binding fragment thereof comprises: VH-CDR1 comprising the amino acid sequence of SEQ ID NO:21 or a variant thereof comprising up to about 3 (such as about any of 1, 2, or 3) amino acid substitutions; and VL-CDR1: SEQ ID NO:29, or a variant thereof comprising up to about 3 (such as about any of 1, 2, or 3) amino acid substitutions. In some embodiments, the anti-YKL-40 antibody or an antigen-binding fragment thereof comprises: VH-CDR1 comprising the amino acid sequence of SEQ ID NO:21; and VL-CDR1: SEQ ID NO:29.

In some embodiments, the anti-YKL-40 antibody or antigen-binding fragment thereof comprises: VH-CDR2 comprising the amino acid sequence of SEQ ID NO:22, or a variant thereof comprising up to about 3 (such as about any of 1, 2, or 3) amino acid substitutions; and VL-CDR2: SEQ ID NO:27, or a variant thereof comprising up to about 3 (such as about any of 1, 2, or 3) amino acid substitutions. In some embodiments, the anti-YKL-40 antibody or an antigen-binding fragment thereof comprises: VH-CDR2 comprising the amino acid sequence of SEQ ID NO:22; and VL-CDR2: SEQ ID NO:27.

In some embodiments, the anti-YKL-40 antibody or antigen-binding fragment thereof comprises: VH-CDR3 comprising the amino acid sequence of SEQ ID NO:23, or a variant thereof comprising up to about 3 (such as about any of 1, 2, or 3) amino acid substitutions; and VL-CDR3: SEQ ID NO:28, or a variant thereof comprising up to about 3 (such as about any of 1, 2, or 3) amino acid substitutions. In some embodiments, the anti-YKL-40 antibody or an antigen-binding fragment thereof comprises: VH-CDR3 comprising the amino acid sequence of SEQ ID NO:23; and VL-CDR3: SEQ ID NO:28.

In some embodiments, the anti-YKL-40 antibody or antigen-binding fragment thereof comprises: VH-CDR1 comprising the amino acid sequence of SEQ ID NO:21, or a variant thereof comprising up to about 3 (such as about any of 1, 2, or 3) amino acid substitutions; VH-CDR2 comprising the amino acid sequence of SEQ ID NO:22, or a variant thereof comprising up to about 3 (such as about any of 1, 2, or 3) amino acid substitutions; VH-CDR3 comprising the amino acid sequence of SEQ ID NO:23, or a variant thereof comprising up to about 3 (such as about any of 1, 2, or 3) amino acid substitutions; VL-CDR1 comprising the amino acid sequence of SEQ ID NO:29, or a variant thereof comprising up to about 3 (such as about any of 1, 2, or 3) amino acid substitutions; VL-CDR2 comprising the amino acid sequence of SEQ ID NO:27, or a variant thereof comprising up to about 3 (such as about any of 1, 2, or 3) amino acid substitutions; and VL-CDR3 comprising the amino acid sequence of SEQ ID NO:28, or a variant thereof comprising up to about 3 (such as about any of 1, 2, or 3) amino acid substitutions.

In one embodiment, the anti-YKL-40 antibody or an antigen-binding fragment thereof comprises at least one of the CDRs selected from the group consisting of: VH-CDR1: SEQ ID NO:24 VH-CDR2: SEQ ID NO:22; VH-CDR3: SEQ ID NO:23; VL-CDR1: SEQ ID NO:29; VL-CDR2: SEQ ID NO:27; and VL-CDR3: SEQ ID NO:28, or a variant or variants thereof. In another embodiment, the anti-YKL-40 antibody comprises all of the CDRs of the group consisting of: VH-CDR1: SEQ ID NO:24 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof.

In some embodiments, the anti-YKL-40 antibody or antigen-binding fragment thereof comprises: VH-CDR1 comprising the amino acid sequence of SEQ ID NO:24 or a variant thereof comprising up to about 3 (such as about any of 1, 2, or 3) amino acid substitutions; and VL-CDR1: SEQ ID NO:29, or a variant thereof comprising up to about 3 (such as about any of 1, 2, or 3) amino acid substitutions. In some embodiments, the anti-YKL-40 antibody or an antigen-binding fragment thereof comprises: VH-CDR1 comprising the amino acid sequence of SEQ ID NO:24; and VL-CDR1: SEQ ID NO:29.

In some embodiments, the anti-YKL-40 antibody or antigen-binding fragment thereof comprises: VH-CDR2 comprising the amino acid sequence of SEQ ID NO:22, or a variant thereof comprising up to about 3 (such as about any of 1, 2, or 3) amino acid substitutions; and VL-CDR2: SEQ ID NO:27, or a variant thereof comprising up to about 3 (such as about any of 1, 2, or 3) amino acid substitutions. In some embodiments, the anti-YKL-40 antibody or an antigen-binding fragment thereof comprises: VH-CDR2 comprising the amino acid sequence of SEQ ID NO:22; and VL-CDR2: SEQ ID NO:27.

In some embodiments, the anti-YKL-40 antibody or antigen-binding fragment thereof comprises: VH-CDR3 comprising the amino acid sequence of SEQ ID NO:23, or a variant thereof comprising up to about 3 (such as about any of 1, 2, or 3) amino acid substitutions; and VL-CDR3: SEQ ID NO:28, or a variant thereof comprising up to about 3 (such as about any of 1, 2, or 3) amino acid substitutions. In some embodiments, the anti-YKL-40 antibody or an antigen-binding fragment thereof comprises: VH-CDR3 comprising the amino acid sequence of SEQ ID NO:23; and VL-CDR3: SEQ ID NO:28.

In some embodiments, the anti-YKL-40 antibody or antigen-binding fragment thereof comprises: VH-CDR1 comprising the amino acid sequence of SEQ ID NO:24, or a variant thereof comprising up to about 3 (such as about any of 1, 2, or 3) amino acid substitutions; VH-CDR2 comprising the amino acid sequence of SEQ ID NO:22, or a variant thereof comprising up to about 3 (such as about any of 1, 2, or 3) amino acid substitutions; VH-CDR3 comprising the amino acid sequence of SEQ ID NO:23, or a variant thereof comprising up to about 3 (such as about any of 1, 2, or 3) amino acid substitutions; VL-CDR1 comprising the amino acid sequence of SEQ ID NO:29, or a variant thereof comprising up to about 3 (such as about any of 1, 2, or 3) amino acid substitutions; VL-CDR2 comprising the amino acid sequence of SEQ ID NO:27, or a variant thereof comprising up to about 3 (such as about any of 1, 2, or 3) amino acid substitutions; and VL-CDR3 comprising the amino acid sequence of SEQ ID NO:28, or a variant thereof comprising up to about 3 (such as about any of 1, 2, or 3) amino acid substitutions.

In one embodiment, the anti-YKL-40 antibody or an antigen-binding fragment thereof comprises at least one of the CDRs selected from the group consisting of: VH-CDR1: SEQ ID NO:25 VH-CDR2: SEQ ID NO:22; VH-CDR3: SEQ ID NO:23; VL-CDR1: SEQ ID NO:29; VL-CDR2: SEQ ID NO:27; and VL-CDR3: SEQ ID NO:28, or a variant or variants thereof. In another embodiment, the anti-YKL-40 antibody comprises all of the CDRs of the group consisting of: VH-CDR1: SEQ ID NO:25 VH-CDR2: SEQ ID NO:22; VH-CDR3: SEQ ID NO:23; VL-CDR1: SEQ ID NO:26; VL-CDR2: SEQ ID NO:27; and VL-CDR3: SEQ ID NO:28, or a variant or variants thereof.

In some embodiments, the anti-YKL-40 antibody or antigen-binding fragment thereof comprises: VH-CDR1 comprising the amino acid sequence of SEQ ID NO:25 or a variant thereof comprising up to about 3 (such as about any of 1, 2, or 3) amino acid substitutions; and VL-CDR1: SEQ ID NO:29, or a variant thereof comprising up to about 3 (such as about any of 1, 2, or 3) amino acid substitutions. In some embodiments, the anti-YKL-40 antibody or an antigen-binding fragment thereof comprises: VH-CDR1 comprising the amino acid sequence of SEQ ID NO:25; and VL-CDR1: SEQ ID NO:29.

In some embodiments, the anti-YKL-40 antibody or antigen-binding fragment thereof comprises: VH-CDR2 comprising the amino acid sequence of SEQ ID NO:22, or a variant thereof comprising up to about 3 (such as about any of 1, 2, or 3) amino acid substitutions; and VL-CDR2: SEQ ID NO:27, or a variant thereof comprising up to about 3 (such as about any of 1, 2, or 3) amino acid substitutions. In some embodiments, the anti-YKL-40 antibody or an antigen-binding fragment thereof comprises: VH-CDR2 comprising the amino acid sequence of SEQ ID NO:22; and VL-CDR2: SEQ ID NO:27.

In some embodiments, the anti-YKL-40 antibody or antigen-binding fragment thereof comprises: VH-CDR3 comprising the amino acid sequence of SEQ ID NO:23, or a variant thereof comprising up to about 3 (such as about any of 1, 2, or 3) amino acid substitutions; and VL-CDR3: SEQ ID NO:28, or a variant thereof comprising up to about 3 (such as about any of 1, 2, or 3) amino acid substitutions. In some embodiments, the anti-YKL-40 antibody or an antigen-binding fragment thereof comprises: VH-CDR3 comprising the amino acid sequence of SEQ ID NO:23; and VL-CDR3: SEQ ID NO:28.

In some embodiments, the anti-YKL-40 antibody or antigen-binding fragment thereof comprises: VH-CDR1 comprising the amino acid sequence of SEQ ID NO:25, or a variant thereof comprising up to about 3 (such as about any of 1, 2, or 3) amino acid substitutions; VH-CDR2 comprising the amino acid sequence of SEQ ID NO:22, or a variant thereof comprising up to about 3 (such as about any of 1, 2, or 3) amino acid substitutions; VH-CDR3 comprising the amino acid sequence of SEQ ID NO:23, or a variant thereof comprising up to about 3 (such as about any of 1, 2, or 3) amino acid substitutions; VL-CDR1 comprising the amino acid sequence of SEQ ID NO:29, or a variant thereof comprising up to about 3 (such as about any of 1, 2, or 3) amino acid substitutions; VL-CDR2 comprising the amino acid sequence of SEQ ID NO:27, or a variant thereof comprising up to about 3 (such as about any of 1, 2, or 3) amino acid substitutions; and VL-CDR3 comprising the amino acid sequence of SEQ ID NO:28, or a variant thereof comprising up to about 3 (such as about any of 1, 2, or 3) amino acid substitutions.

In some embodiments, the antibody or the antibody fragment is modified. In some embodiments, the modifications include fusion of the antibody or the antigen-binding fragment thereof with portions of another protein, or a protein fragment. In some embodiments, the antibody or the antibody fragment thereof of the invention is modified to increase the circulating half-life of the same in vivo. For example, the antibody of the fragment may be fused with an FcRn molecule, which is also known as neonatal Fc receptor to stabilize the antibody in vivo. (Nature Reviews Immunology 7:715-725)

In some embodiments, the invention includes human-YKL-40 binding single chain variable fragment (scFv). An scFv may comprise heavy chain variable region sequences designated in SEQ ID NOs 1-12, and light chain variable regions designated in SEQ ID NOs 13-20. An scFv may comprise heavy chain variable region sequences designated in SEQ ID NOs 1-12, and light chain variable regions designated SEQ ID NOs 13-20. CDR sequences incorporated within the scFv having amino acid sequence identity of 80%, 81%, 82%, 83%, 84%, 85% 86%, 87%, 88%, 89%, 90%, 91%, 92%, 93%, 94%, 95%, 96%, 97%, 98%, and 99% to the sequences described in the present disclosure are encompassed within the scope of the present disclosure.

In some embodiments, the antibody is conjugated to an “effector” moiety. The effector moiety can be any number of molecules, including labeling moieties such as radioactive labels, fluorescent labels or other suitable detectable marker, or can be a therapeutic moiety. In one aspect the antibody modulates the activity of the protein. Such effector moieties include, but are not limited to, an anti-tumor drug, a toxin, a radioactive agent, a probe for detecting the localization of the antibody, a cytokine, a second antibody or an enzyme. Further, the invention provides an embodiment wherein the antibody of the invention is linked to an enzyme that converts a prodrug into a cytotoxic agent.

The immunoconjugate, that is, the antibody conjugated to an effector moeity, can be used for targeting the effector moiety to a YKL-40 positive cell, particularly cells, which express the YKL-40 protein. Such differences can be readily apparent when viewing the bands of gels with approximately similarly loaded with test and controls samples. Examples of cytotoxic agents include, but are not limited to ricin, doxorubicin, daunorubicin, taxol, ethiduim bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

In some embodiments, the invention provides antibodies to YKL-40. YKL-40 antibodies may be used systemically to treat cancer (e.g., melanoma, metastatic cancer, metastatic melanoma) alone or when conjugated with an effector moiety. YKL-40 antibodies conjugated with toxic agents, such as ricin, as well as unconjugated antibodies may be useful therapeutic agents naturally targeted to YKL-40-bearing cancer cells. Such antibodies can be useful in blocking cancer progression and invasiveness. In some embodiments, immunoconjugates are prepared using means well known to a skilled artisan.

### Biomarker

A biomarker is a biomolecule that is differentially present in a sample taken from an individual of one phenotypic status (e.g., having a disease) as compared with an individual of another phenotypic status (e.g., not having the disease). A biomarker is differentially present between the two individuals if the mean or median expression level of the biomarker in the different individuals is calculated to be statistically significant. Biomarkers, alone or in combination, provide measures of relative risk that an individual belongs to one phenotypic status or another. Therefore, they are useful as markers for diagnosis of disease, for assessing the severity of disease, for assessing therapeutic effectiveness of a drug, and for assessing drug toxicity.

Accordingly, the invention provides methods for identifying one or more biomarkers which can be used to aid in the diagnosis, detection, and prediction of an inflammatory disease or disorder. The methods of the invention are carried out by obtaining a set of measured values for a plurality of biomarkers from a biological sample derived from a test individual, obtaining a set of measured values for a plurality of biomarkers from a biological sample derived from a control individual, comparing the measured values for each biomarker between the test and control sample, and identifying biomarkers which are significantly different between the test value and the control value, also referred to as a reference value.

The process of comparing a measured value and a reference value can be carried out in any convenient manner appropriate to the type of measured value and reference value for the biomarker of the invention. For example, “measuring” can be performed using quantitative or qualitative measurement techniques, and the mode of comparing a measured value and a reference value can vary depending on the measurement technology employed. For example, when a qualitative calorimetric assay is used to measure biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the invention will most commonly be quantitative values (e.g., quantitative measurements of concentration). In other examples, measured values are qualitative. As with qualitative measurements, the comparison can be made by inspecting the numerical data, or by inspecting representations of the data (e.g., inspecting graphical representations such as bar or line graphs).

A measured value is generally considered to be substantially equal to or greater than a reference value if it is at least about 95% of the value of the reference value. A measured value is considered less than a reference value if the measured value is less than about 95% of the reference value. A measured value is considered more than a reference value if the measured value is at least more than about 5% greater than the reference value.

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a desired biomarker. Alternately, a separate device (e.g., a digital computer) may be used to compare the measured value(s) and the reference value(s). Automated devices for comparison may include stored reference values for the biomarker(s) being measured, or they may compare the measured value(s) with reference values that are derived from contemporaneously measured reference samples.

### Diagnostic

The invention relates in part to the discovery that levels of chitinase-like family of secreted proteins (CLPs) are markedly increased during inflammation, lung inflammation, onset of asthma, onset of lung disease, onset of lung cancer, and the like, correlating with upregulated lung expression of the mRNA for Chi3l1 that peaks during the time of lung repair. In addition, the level of lung Chi3l1 expression and Brp-39 (YKL-40) excretion directly correlates with lung disease severity, inflammation and fibrosis.

The present invention also relates partly to the discovery that upregulation of chitinase 3-like 1/Brp-39/YKL-40 in response to ischemic injury is important in inhibiting cell apoptosis in vivo and that the chitinase 3-like 1/Brp-39/YKL-40 pathway serves to limit the severity of injury and maintain sufficient lung function to keep the subject alive and promote proliferation of viable cells to effect subsequent lung repair. In some embodiments, the present invention relates to the discovery that the chitinase 3-like 1/Brp-39/YKL-40 pathway serves to limit the severity of injury and maintain sufficient organ function to keep the subject alive and promote proliferation of viable cells to effect subsequent organ repair wherein the organ may include the kidney, liver, heart, pancreas, and others.

Accordingly, the present invention relates generally to diagnostic methods and markers, prognostic methods and markers, and therapy evaluators for lung diseases and disorders, such as asthma, lung disease, lung cancer, and other diseases or disorders that are associated with lung inflammation. In some embodiments, the present invention relates to diagnostic methods and markers, prognostic methods and markers, and therapy evaluators for diseases and disorders, such as diseases, such as inflammatory diseases, of organs including those of the kidney, liver, heart, pancreas, among others.

In certain embodiments, the method comprises the step of obtaining a sample from the subject, and assessing the level of chitinase 3-like 1/Brp-39/YKL-40 in the sample. Thus, the present invention relates to markers for determining lung inflammation status in a subject, methods for diagnosis of an inflammatory disease or disorder, methods of determining predisposition to an inflammatory disease or disorder, methods of monitoring progression/regression of an inflammatory disease or disorder, methods of assessing efficacy of compositions for treating an inflammatory disease or disorder, methods of screening compositions for activity in modulating markers of an inflammatory disease or disorder, methods of treating an inflammatory disease or disorder, as well as other methods based on markers of an inflammatory disease or disorder. In some embodiments, the methods include detecting and/or measuring free and drug-bound levels of chitinase 3-like 1/Brp-39/YKL-40 in the sample. In some embodiments, the sample is a blood sample, serum sample, lymph sample, urine sample, or other sample obtained from a subject.

In some embodiments, the inflammatory disease or disorder is an inflammatory disease or disorder of the lung. In some embodiments, the inflammatory disease or disorder is inflammation in the lung. In some embodiments, the inflammatory disease or disorder is asthma. In some embodiments, the inflammatory disorder is cancer. In some embodiments, the cancer is metastatic cancer, such as metastatic melanoma.

In certain embodiments, the invention further provides methods for permitting refinement of disease or disorder diagnosis, disease or disorder risk prediction, and clinical management of individuals associated with an inflammatory disease or disorder. The markers of the invention represent an assay for assessing an inflammatory disease or disorder that can be used for determining the disease or disorder state or disease or disorder risk. The detection of the selective markers of the invention in a subject, or a sample obtained there from, permits refinement of disease or disorder diagnosis, disease or disorder risk prediction, and clinical management of individuals being treated with agents that are associated with an inflammatory disease or disorder.

In one embodiment of the invention provides a method for identifying an individual as having an inflammatory disease or disorder such as a lung injury comprising the steps of detecting or measuring chitinase 3-like 1/Brp-39/YKL-40 in a body sample obtained from an individual diagnosed with an inflammatory or lung injury, or a putative at-risk individual, then comparing the levels of chitinase 3-like 1/Brp-39/YKL-40 present in the test sample to chitinase 3-like 1/Brp-39/YKL-40 levels detected or measured in a comparator, such as a positive control, a negative control, a historical norm, or a sample obtained from one or more otherwise identical, normal, not-at-risk individuals. In some instances, the level of chitinase 3-like 1/Brp-39/YKL-40 expression is compared with an average value obtained from more than one not-at-risk individual. In other instances, the level of chitinase 3-like 1/Brp-39/YKL-40 expression is compared with chitinase 3-like 1/Brp-39/YKL-40 assessed in a sample obtained from one normal, not-at-risk individual. In yet another instance, the level of chitinase 3-like 1/Brp-39/YKL-40 expression in the putative at-risk individual is compared with the level of chitinase 3-like 1/Brp-39/YKL-40 expression in a sample obtained from the same individual at a different time.

In another embodiment, a method of diagnosing an inflammatory disorder in a subject comprises the steps of obtaining a first sample from the subject at a first time; assessing the level of a marker of the invention in the first sample to obtain a baseline level; obtaining a second sample from the subject at a second time and assessing the level of the marker in the second sample to obtain a second level. If the second level is significantly higher compared to the baseline level, the subject is at an increased risk of developing or having an inflammatory disorder. In one embodiment, the second level is also compared to a comparator, such as a positive control, a negative control, a historical norm, or a reference population of a subject without the disorder; if the second level is significantly higher compared to the level derived from the comparator, the subject is at an increased risk of developing or having an inflammatory disorder.

A biomarker is typically a protein, found in a bodily fluid, the level of which varies with disease state and can be quantified. The quantified level can then be compared to a known value. The comparison can be used for a variety of purposes, including but not limited to, diagnosis of an inflammatory disease or disorder, prognosis of an inflammatory disease or disorder, and monitoring treatment of an inflammatory disease or disorder.

In some embodiments, the invention provides a kit with a detection reagent which binds to chitinase 3-like 1/Brp-39/YKL-40, fragments, analogs, metabolites, or other analytes.

In some embodiments, a detection reagent is immobilized on a solid matrix such as a porous strip or bead to form at least one inflammatory injury biomarker detection site.

In various embodiments, the levels of a biomarker of the invention are assessed in various biological samples, for example bodily fluids. Non-limiting examples of bodily fluid include whole blood, plasma, serum, bile, lymph, pleural fluid, semen, saliva, sputum, sweat, urine, and cerebral spinal fluid. In one embodiment, the bodily fluid is selected from the group of whole blood, plasma, and serum. In another embodiment, the bodily fluid is whole blood. In yet another embodiment, the bodily fluid is plasma. In still yet another embodiment, the bodily fluid is serum.

In some embodiments, the bodily fluid is obtained from the individual using conventional methods in the art. For instance, one skilled in the art knows how to draw blood or sputum and how to process it in order to obtain serum and/or plasma and/or protein fraction for use in the method. Generally speaking, the method maintains the integrity of the biomarkers of the invention such that it can be accurately quantified in the bodily fluid. Methods for collecting blood, sputum or fractions thereof are well known in the art. For example, see U.S. Pat. No. 5,286,262, which is hereby incorporated by reference in its entirety.

In another embodiment, the sample is a tissue sample or blood sample, and the blood sample may be a (blood) serum or (blood) plasma sample. In some embodiments, the sample is a sputum sample.

In various embodiments, assessment of biomarker levels encompasses assessment of the level of protein concentration and/or the level of enzymatic activity of the biomarker. In either case, the level is quantified such that a value, an average value, or a range of values is determined. In one embodiment, the level of protein concentration of the inflammatory disorder biomarker is quantified.

There are numerous known methods and kits for measuring the amount or concentration of a protein in a sample, including as non-limiting examples, ELISA, western blot, absorption measurement, colorimetric determination, Lowry assay, Bicinchoninic acid assay, or a Bradford assay. Commercial kits include ProteoQwest™ Colormetric Western Blotting Kits (Sigma-Aldrich, Co.), QuantiPro™ bicinchoninic acid (BCA) Protein Assay Kit (Sigma-Aldrich, Co.), FluoroProfile™ Protein Quantification Kit (Sigma-Aldrich, Co.), the Coomassie Plus—The Better Bradford Assay (Pierce Biotechnology, Inc.), and the Modified Lowry Protein Assay Kit (Pierce Biotechnology, Inc.). In certain embodiments, the protein concentration is measured using a luminex based multiplex immunoassay panel. However, the invention should not be limited to any particular assay for assessing the level of a biomarker of the invention. That is, any currently known assay used to detect protein levels and assays to be discovered in the future can be used to detect the biomarkers of the invention.

Methods of quantitatively assessing the level of a protein in a biological sample such as blood, serum and plasma are well known in the art. In some embodiments, assessing the level of a protein involves the use of a detector molecule for the biomarker. Detector molecules can be obtained from commercial vendors or can be prepared using conventional methods available in the art. Exemplary detector molecules include, but are not limited to, an antibody that binds specifically to the biomarker, a naturally-occurring cognate receptor, or functional domain thereof, for the biomarker, or a small molecule that binds specifically to the biomarker.

In another embodiment, the level of a biomarker is assessed using an antibody, such as an antibody or combination of antibodies as described herein. Thus, non-limiting exemplary methods for assessing the level of a biomarker in a biological sample include various immunoassays, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY.

Other methods for assessing the level of a protein include chromatography (e.g., HPLC, gas chromatography, liquid chromatography) and mass spectrometry (e.g., MS, MS-MS). For instance, a chromatography medium comprising a cognate receptor for the biomarker or a small molecule that binds to the biomarker can be used to substantially isolate the biomarker from the biological sample. Small molecules that bind specifically to a biomarker can be identified using conventional methods in the art, for instance, screening of compounds using combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection.

The level of a substantially isolated protein can be quantitated directly or indirectly using a conventional technique in the art such as spectrometry, Bradford protein assay, Lowry protein assay, biuret protein assay, or bicinchoninic acid protein assay, as well as immunodetection methods.

In another embodiment, the level of enzymatic activity of the biomarker is quantified. Generally, enzyme activity may be measured by means known in the art, such as measurement of product formation, substrate degradation, or substrate concentration, at a selected point(s) or time(s) in the enzymatic reaction. There are numerous known methods and kits for measuring enzyme activity. For example, see U.S. Pat. No. 5,654,152. Some methods may require purification of the biomarker prior to measuring the enzymatic activity of the biomarker. A pure biomarker constitutes at least about 90%, preferably, 95% and even more preferably, at least about 99% by weight of the total protein in a given sample. Biomarkers of the invention may be purified according to methods known in the art, including, but not limited to, ion-exchange chromatography, size-exclusion chromatography, affinity chromatography, differential solubility, differential centrifugation, and HPLC.

As apparent from the examples disclosed herein, diagnostic tests that use the biomarkers of the invention exhibit a sensitivity and specificity of at least 75%, at least 80%, at least 85%, at least 90%, at least 95%, at least 98% and about 100%. In some instances, screening tools of the present invention exhibits a high sensitivity of at least 75%, at least 80%, at least 85%, at least 90%, at least 95%, at least 98% and about 100%. Without wishing to be bound by any particular theory, it is believed that screening tools should exhibit high sensitivity, but specificity can be low. However, diagnostics should have high sensitivity and specificity.

Determining inflammatory disease or disorder status typically involves classifying an individual into one of two or more groups based on the results of the diagnostic test. The diagnostic tests described herein can be used to classify an individual into a number of different states. In one embodiment, the invention provides methods for determining the presence or absence of an inflammatory disease in an individual (status: disease v. non-disease). The presence or absence of inflammatory disease is determined by measuring the relevant biomarker or biomarkers in samples obtained from individuals and then either submitting them to a classification algorithm or comparing them with a reference amount and/or pattern of biomarkers that is associated with the particular risk level.

In another embodiment, the invention provides methods for determining the risk of developing disease in an individual. Biomarker amounts or patterns are characteristic of various risk states, e.g., high, medium or low. The risk of developing an inflammatory disease is determined by measuring the relevant biomarker or biomarkers in sample obtained from individuals and then either submitting them to a classification algorithm or comparing them with a reference amount and/or pattern of biomarkers that is associated with the particular risk level.

In yet another embodiment, the invention provides methods for determining the stage of a disease in an individual. Each stage of the disease can be characterized by the amount of a biomarker or relative amounts of a set of biomarkers (i.e., a pattern) that are found in a sample obtained from the individual. The stage of disease is determined by measuring the relevant biomarker or biomarkers and then either submitting them to a classification algorithm or comparing them with a reference amount and/or pattern of biomarkers that is associated with the particular stage.

In another embodiment, the invention provides methods for determining the course of disease in an individual. Disease course refers to changes in disease status over time, including disease progression (worsening) and disease regression (improvement). Over time, the amounts or relative amounts (e.g., the pattern) of the biomarkers changes. For example, levels of various biomarkers of the present invention increase with progression of disease. Accordingly, this method involves measuring the level of one or more biomarkers in an individual at two or more different time points, e.g., a first time and a second time, and comparing the change in amounts. The course of disease is determined based on these comparisons.

In some instances, the levels of various biomarkers of the invention decreases with disease progression. In this method, the level of one or more biomarkers in a sample from an individual is measured at two or more different time points, e.g., a first time and a second time, and the change in levels, if any is assessed. The course of disease is determined based on these comparisons.

Similarly, changes in the rate of disease progression (or regression) may be monitored by measuring the level of one or more biomarkers at different times and calculating the rate of change in biomarker levels. The ability to measure disease state or rate of disease progression is important for drug treatment studies where the goal is to slow down or arrest disease progression using therapy.

Additional embodiments of the invention relate to the communication of the results or diagnoses or both to technicians, physicians or patients, for example. In certain embodiments, computers are used to communicate results or diagnoses or both to interested parties, e.g., physicians and their patients.

In certain embodiments, the methods of the invention further comprise managing individual treatment based upon their disease status. Such management includes the actions of the physician or clinician subsequent to determining disease status. For example, if a physician makes a diagnosis of an inflammatory disease, then a certain regime of treatment, such as prescription or administration of the therapeutic compound might follow. Alternatively, a diagnosis of a non-disease might be followed by further testing to determine any other diseases that the patient might be suffering from. Also, if the test is inconclusive with respect to an inflammatory disease status, further tests may be called for.

In another embodiment of the invention, a diagnosis based on the presence or absence or relative levels in the biological sample of an individual of the relevant biomarkers disclosed herein is communicated to the individual as soon as possible after the diagnosis is obtained.

According to yet another aspect, the present invention provides a method of assessing efficacy of a treatment of an inflammatory disease in a patient comprising: a) determining a baseline level of biomarkers in a first sample obtained from the patient before receiving the treatment; b) determining the level of same biomarkers in a second sample obtained from the patient after receiving the treatment; wherein an increase in the levels of the biomarkers in the post-treatment sample is correlated with a positive treatment outcome.

### Detection Methods

Any methods available in the art for identification or detection of chitinase 3-like 1/Brp-39/YKL-40 are encompassed herein. Chitinase 3-like 1/Brp-39/YKL-40 can be detected at a nucleic acid level or a protein level. In order to determine up-regulation of chitinase 3-like 1/Brp-39/YKL-40 expression, levels of the chitinase 3-like 1/Brp-39/YKL-40 are measured in the body sample to be examined and compared with a corresponding body sample that originates from a normal, not-at-risk individual. In another embodiment of the invention, up-regulation of chitinase 3-like 1/Brp-39/YKL-40 is determined by measuring levels of chitinase 3-like 1/Brp-39/YKL-40 in the body sample to be examined and comparing with an average value obtained from more than one not-at-risk individual. In still another embodiment of the invention, up-regulation of chitinase 3-like 1/Brp-39/YKL-40 is determined by measuring levels of chitinase 3-like 1/Brp-39/YKL-40 in the body sample to be examined and comparing with levels of chitinase 3-like 1/Brp-39/YKL-40 obtained from a body sample obtained from the same individual at a different time.

Methods for detecting chitinase 3-like 1/Brp-39/YKL-40 comprise any method that determines the presence, quantity or activity of chitinase 3-like 1/Brp-39/YKL-40 either at the nucleic acid or protein level. Such methods are well known in the art and include but are not limited to western blots, northern blots, southern blots, ELISA, immunoprecipitation, immunofluorescence, flow cytometry, immunocytochemistry, nucleic acid hybridization techniques, nucleic acid reverse transcription methods, and nucleic acid amplification methods. In particular embodiments, chitinase 3-like 1/Brp-39/YKL-40 is detected on a protein level using, for example, antibodies that are directed against chitinase 3-like 1/Brp-39/YKL-40 protein. These antibodies can be used in various methods such as Western blot, ELISA, immunoprecipitation, or immunocytochemistry techniques.

The invention should not be limited to any one method of protein or nucleic acid detection method recited herein, but rather should encompass all known or heretofore unknown methods of detection as are, or become, known in the art.

Samples may need to be modified in order to render the chitinase 3-like 1/Brp-39/YKL-40 antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding, for example binding of one or more antibodies such as those described herein, may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO 51699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a chitinase 3-like 1/Brp-39/YKL-40 is then incubated with the sample.

Techniques for detecting antibody binding are well known in the art. Antibody binding to chitinase 3-like 1/Brp-39/YKL-40 may be detected through the use of chemical reagents that generate a detectable signal that corresponds to the level of antibody binding and, accordingly, to the level of chitinase 3-like 1/Brp-39/YKL-40 protein expression. In some immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+system (Dako North America, Inc., Carpinteria, Calif.) and Mach 3 system (Biocare Medical, Walnut Creek, Calif.), may be used to practice the present invention.

In one particular immunocytochemistry method of the invention, antibody binding to a biomarker is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., biomarker overexpression). Alternatively, samples may be reviewed via automated microscopy or by personnel with the assistance of computer software that facilitates the identification of positive staining cells.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the invention, there also exist in the art video-microscopy and software methods for the quantitative determination of an amount of multiple molecular species (e.g., biomarker proteins) in a biological sample, wherein each molecular species present is indicated by a representative dye marker having a specific color. Such methods are also known in the art as colorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 and U.S. patent application Ser. No. 10/057,729 to Marcelpoil, incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for chitinase 3-like 1/Brp-39/YKL-40 protein. Methods for making antibodies and for selecting appropriate antibodies are known in the art. See, for example, Celis, J. E. ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined in reference to the type of biological sample being tested. In particular embodiments, appropriate antibody titers for use cytology preparations are determined by initially testing various antibody dilutions on formalin-fixed paraffin-embedded normal tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody is then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the invention will vary depending on such factors as time for binding, level of specificity of the antibody for the chitinase 3-like 1/Brp-39/YKL-40 protein, and method of body sample preparation. Furthermore, the detection chemistry used to visualize antibody binding to a chitinase 3-like 1/Brp-39/YKL-40 protein must also be optimized to produce the desired signal to noise ratio.

Immunoassays

Immunoassays, in their simplest and most direct sense, are binding assays. Some immunoassays are the various types of enzyme linked immunosorbent assays (ELISA) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the chitinase 3-like 1/Brp-39/YKL-40 proteins of the invention are immobilized onto a selected surface exhibiting protein affinity, such as a well in a polystyrene microtiter plate. Then, a test composition suspected of containing the biomarker antigen, such as a clinical sample, is added to the wells. After binding and washing to remove non-specifically bound immune complexes, the bound antibody may be detected. Detection is generally achieved by the addition of a second antibody specific for the target protein that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the chitinase 3-like 1/Brp-39/YKL-40 antigen are immobilized onto the well surface and then contacted with the antibodies of the invention. After binding and washing to remove non-specifically bound immune complexes, the bound antigen is detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the chitinase 3-like 1/Brp-39/YKL-40, and detected by means of their label. The amount of marker antigen in an unknown sample is then determined by mixing the sample with the labeled antibodies before or during incubation with coated wells. The presence of chitinase 3-like 1/Brp-39/YKL-40 antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immune complexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. One washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immune complex can be detected with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

Nucleic Acid-Based Techniques

In other embodiments, the expression of chitinase 3-like 1/Brp-39/YKL-40 is detected at the nucleic acid level. Nucleic acid-based techniques for assessing expression are well known in the art and include, for example, determining the level of chitinase 3-like 1/Brp-39/YKL-40 mRNA in a body sample. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

The term “probe” refers to any molecule that is capable of selectively binding to a specifically intended target biomolecule, for example, a nucleotide transcript or a protein encoded by or corresponding to chitinase 3-like 1/Brp-39/YKL-40. Probes can be synthesized by one of skill in the art, or derived from appropriate biological preparations. Probes may be specifically designed to be labeled with a detectable label. Examples of molecules that can be used as probes include, but are not limited to, RNA, DNA, proteins, antibodies, and organic molecules.

Isolated mRNA can be detected in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding chitinase 3-like 1/Brp-39/YKL-40 of the present invention. Hybridization of an mRNA with the probe indicates that the chitinase 3-like 1/Brp-39/YKL-40 in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

An alternative method for determining the level of chitinase 3-like 1/Brp-39/YKL-40 mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self-sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), and rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan™ System). Such methods typically use pairs of oligonucleotide primers that are specific for the biomarker of interest. Methods for designing oligonucleotide primers specific for a known sequence are well known in the art.

Chitinase 3-like 1/Brp-39/YKL-40 expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

### Treatment

The disclosure presented herein demonstrates that upregulation of YKL-40 plays a key role in promoting cell proliferation in vivo, and that this pathway serves to promote the severity of tissue injury in disease and disorders, including inflammatory diseases and disorders and cancer (e.g., primary cancers, metastatic cancers, asthma). Furthermore, the disclosure as presented demonstrates that blocking YKL-40 protein significantly attenuates inflammation associated with inflammatory diseases and disorders (e.g., cancer, primary cancers, metastatic cancers, asthma), and that by negatively regulating YKL-40 protein by means such as through the use of antibodies such as those disclosed herein, wherein in some embodiments, the negative effects of the associated inflammation are mitigated, attenuated, or lessened.

The invention provides compositions and methods useful in treating an individual diagnosed with an inflammatory disease or disorder (e.g., asthma, cancer) by the administration of one of more of the anti-YKL-40 antibodies described herein. In various embodiments, the antibody is administered locally, regionally, or systemically. Treating an individual diagnosed with an inflammatory disease or disorder encompasses a method of inhibiting the progression of the inflammatory disease or disorder in an individual diagnosed with an inflammatory disease or disorder. By “inhibiting the progression of an inflammatory disorder” is intended to mean that the progressive histological or morphometric changes associated with the clinical sequelae of an inflammatory disease or disorder, for example infiltration of inflammatory cells, or cell proliferation is halted, prevented, diminished or attenuated. It will be appreciated that the method of the present invention may also be to practiced in an individual at risk of developing an inflammatory disease or disorder whereby an individual identified as being at risk of developing an inflammatory disease or disorder may be prevented from developing that would subsequently lead to a clinical manifestation of an inflammatory disease or disorder.

Treating an individual diagnosed with cancer encompasses administering at least one of the anti-YKL-40 antibodies described herein, to an individual diagnosed with cancer, such that the cancer is halted, prevented, diminished, or attenuated. It will be appreciated that the method of the present invention may also be practiced in an individual at risk of developing cancer whereby an individual identified as being at risk of developing cancer may be prevented from developing that would subsequently lead to a clinical manifestation of cancer. The following are non-limiting examples of cancers that can be treated by the disclosed methods and compositions: acute lymphoblastic, acute myeloid leukemia, adrenocortical carcinoma, adrenocortical carcinoma, childhood, appendix cancer, basal cell carcinoma, bile duct cancer, extrahepatic, bladder cancer, bone cancer, osteosarcoma and malignant fibrous histiocytoma, brain stem glioma, childhood, brain tumor, adult, brain tumor, brain stem glioma, childhood, brain tumor, central nervous system atypical teratoid/rhabdoid tumor, childhood, central nervous system embryonal tumors, cerebellar astrocytoma, cerebral astrocytotna/malignant glioma, craniopharyngioma, ependymoblastoma, ependymoma, medulloblastoma, medulloepithelioma, pineal parenchymal tumors of intermediate differentiation, supratentorial primitive neuroectodermal tumors and pineoblastoma, visual pathway and hypothalamic glioma, brain and spinal cord tumors, breast cancer, bronchial tumors, Burkitt lymphoma, carcinoid tumor, carcinoid tumor, gastrointestinal, central nervous system atypical teratoid/rhabdoid tumor, central nervous system embryonal tumors, central nervous system lymphoma, cerebellar astrocytoma cerebral astrocytoma/malignant glioma, childhood, cervical cancer, chordoma, chronic lymphocytic leukemia, chronic myelogenous leukemia, chronic myeloproliferative disorders, colon cancer, colorectal cancer, craniopharyngioma, cutaneous T-cell lymphoma, esophageal cancer, ewing family of tumors, extragonadal germ cell tumor, extrahepatic bile duct cancer, eye cancer, intraocular melanoma, eye cancer, retinoblastoma, gallbladder cancer, gastric (stomach) cancer, gastrointestinal carcinoid tumor, gastrointestinal stromal tumor (gist), germ cell tumor, extracranial, germ cell tumor, extragonadal, germ cell tumor, ovarian, gestational trophoblastic tumor, glioma, glioma, brain stem, glioma, cerebral astrocytoma, glioma, visual pathway and hypothalamic, hairy cell leukemia, head and neck cancer, hepatocellular (liver) cancer, histiocytosis, langerhans cell, Hodgkin lymphoma, hypopharyngeal cancer, hypothalamic and visual pathway glioma, intraocular melanoma, islet cell tumors, kidney (renal cell) cancer, langerhans cell histiocytosis, laryngeal cancer, leukemia, acute lymphoblastic, leukemia, acute myeloid, leukemia, chronic lymphocytic, leukemia, chronic myelogenous, leukemia, hairy cell, lip and oral cavity cancer, liver cancer, lung cancer, non-small cell, lung cancer, small cell, lymphoma, aids-related, lymphoma, Burkitt lymphoma, cutaneous T-cell, non-Hodgkin lymphoma, primary central nervous system, macroglobulinemia, Waldenstrom, malignant fibrous histiocvtoma of bone and osteosarcoma, medulloblastoma, melanoma, melanoma, intraocular (eye), Merkel cell carcinoma, mesothelioma, metastatic squamous neck cancer with occult primary, mouth cancer, multiple endocrine neoplasia syndrome, multiple myeloma/plasma cell neoplasm, mycosis, fungoides, myelodysplastic syndromes, myelodysplastic/myeloproliferative diseases, myelogenous leukemia, myeloid leukemia, multiple myeloma, myeloproliferative disorders, nasal cavity and paranasal sinus cancer, nasopharyngeal cancer, neuroblastoma, non-small cell lung cancer, oral cancer, oral cavity cancer, oropharyngeal cancer, osteosarcoma and malignant fibrous histiocytoma of bone, ovarian cancer, ovarian epithelial cancer, ovarian germ cell tumor, ovarian low malignant potential tumor, pancreatic cancer, islet cell tumors, papillomatosis, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, renal pelvis and ureter, transitional cell cancer, respiratory tract carcinoma, retinoblastoma, rhabdomyosarcoma, salivary gland cancer, sarcoma, Ewing family of tumors, kaposi sarcoma, sarcoma, uterine, sezary syndrome, skin cancer (nonmelanoma), skin cancer (melanoma), skin carcinoma, Merkel cell, small cell lung cancer, small intestine cancer, soft tissue sarcoma, squamous cell carcinoma, squamous neck cancer, stomach (gastric) cancer, supratentorial primitive neuroectodermal tumors, T-cell lymphoma, cutaneous, testicular cancer, throat cancer, thymoma and thymic carcinoma, thyroid cancer, transitional cell cancer of the renal pelvis and ureter, trophoblastic tumor, gestational, urethral cancer, uterine cancer, endometrial, uterine sarcoma, vaginal cancer, vulvar cancer, Waldenstrom macroglobulinemia, and Wilms tumor.

In another embodiment, the invention provides a method to treat cancer metastasis comprising treating the subject prior to, concurrently with, or subsequently to the treatment with one of the anti-YKL-40 antibodies described herein, along with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The anti-YKL-40 antibodies described herein can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

Attenuating, blocking, inhibiting, reducing or lessening chitinase 3-like 1/Brp-39/YKL-40 expression or activity can be accomplished using any method known to the skilled artisan. Examples of methods to decrease chitinase 3-like 1/Brp-39/YKL-40 expression include, but are not limited to decreasing expression of an endogenous chitinase 3-like 1 gene, decreasing expression of chitinase 3-like 1/Brp-39/YKL-40 mRNA, and decreasing expression of chitinase 3-like 1/Brp-39/YKL-40 protein. An agent, composition or compound that reduces or decreases chitinase 3-like 1/Brp-39/YKL-40 expression or activity may be a compound or composition that decreases expression of a chitinase 3-like 1 gene, a compound or composition that decreases chitinase 3-like 1/Brp-39/YKL-40 mRNA half-life, stability and/or expression, or a compound or composition that enhances chitinase 3-like 1/Brp-39/YKL-40 protein function. An agent, composition or compound that reduces or decreases chitinase 3-like 1/Brp-39/YKL-40 expression or activity may be any type of compound, including but not limited to, a polypeptide, a nucleic acid, an aptamer, an antibody, a peptidometic, and a small molecule, or combinations thereof. In various embodiments, the agent, composition or compound that reduces or decreases chitinase 3-like 1/Brp-39/YKL-40 expression or activity is any of the antibodies described herein.

The present invention should in no way be construed to be limited to the inhibitors described herein, but rather should be construed to encompass any inhibitor of chitinase 3-like 1/Brp-39/YKL-40, both known and unknown, that reduces inflammation, infiltration of inflammatory cells to an organ or tissue, or prevents proliferation or metastasis of cancer.

The treatment methods of the invention comprises administering a therapeutically effective amount of an anti-chitinase 3-like 1/Brp-39/YKL-40 antibody such as one or more such antibodies as described herein, or an agent that enhances or increases anti-chitinase 3-like 1/Brp-39/YKL-40 antibody expression or activity to a mammal in need thereof.

Methods of prophylaxis (i.e., prevention or decreased risk of disease), as well as reduction in the frequency or severity of symptoms associated with inflammatory disorder or any related disease or disorder, are encompassed by the present invention. The method of the invention comprises administering a therapeutically effective amount of an anti-chitinase 3-like 1/Brp-39/YKL-40 antibody such as one or more antibodies as described herein, or an agent that reduces or decreases chitinase 3-like 1/Brp-39/YKL-40 expression or activity to a mammal in combination with other therapeutic agents to treat a mammal. An anti-chitinase 3-like 1/Brp-39/YKL-40 antibody or an agent that reduces or decreases chitinase 3-like 1/Brp-39/YKL-40 expression or activity may be administered, before, during, after, or throughout the administration of the therapeutic agent. The compositions and methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone)), steroids (including prednisone and dexamethasone), and antidepressants (including gabapentin, amitriptyline, imipramine, doxepin) antihistamines, antitussives, muscle relaxants, bronchodilators, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like.

The therapeutic and prophylactic methods of the invention thus encompass the use of pharmaceutical compositions comprising one or more anti-chitinase 3-like 1/Brp-39/YKL-40 antibodies, or combinations thereof, as described herein. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

The pharmaceutical compositions useful for practicing the invention may be administered to deliver a dose of at least about 1 ng/kg, at least about 5 ng/kg, at least about 10 ng/kg, at least about 25 ng/kg, at least about 50 ng/kg, at least about 100 ng/kg, at least about 500 ng/kg, at least about 1 □g/kg, at least about 5 □g/kg, at least about 10 □g/kg, at least about 25 □g/kg, at least about 50 □g/kg, at least about 100 □g/kg, at least about 500 g/kg, at least about 1 mg/kg, at least about 5 mg/kg, at least about 10 mg/kg, at least about 25 mg/kg, at least about 50 mg/kg, at least about 100 mg/kg, at least about 200 mg/kg, at least about 300 mg/kg, at least about 400 mg/kg, and at least about 500 mg/kg of body weight of the subject. In one embodiment, the invention administers a dose which results in a concentration of the anti-YKL-40 antibody of the present invention of at least about 1 pM, at least about 10 pM, at least about 100 pM, at least about 1 nM, at least about 10 nM, at least about 100 nM, at least about 1 μM, at least about 2 μM, at least about 3 μM, at least about 4 μM, at least about 5 μM, at least about 6 μM, at least about 7 μM, at least about 8 μM, at least about 9 μM and at least about 10 μM in an individual. In another embodiment, the invention envisions administration of a dose which results in a concentration of the anti-YKL-40 antibody of the present invention between at least about 1 pM, at least about 10 pM, at least about 100 pM, at least about 1 nM, at least about 10 nM, at least about 100 nM, at least about 1 μM, at least about 2 μM, at least about 3 μM, at least about 4 μM, at least about 5 μM, at least about 6 μM, at least about 7 μM, at least about 8 μM, at least about 9 μM and at least about 10 μM in the plasma of an individual.

In some embodiments, the pharmaceutical compositions useful for practicing the invention may be administered to deliver a dose of no more than about 1 ng/kg, no more than about 5 ng/kg, no more than about 10 ng/kg, no more than about 25 ng/kg, no more than about 50 ng/kg, no more than about 100 ng/kg, no more than about 500 ng/kg, no more than about 1 □g/kg, no more than about 5 □g/kg, no more than about 10 □g/kg, no more than about 25 □g/kg, no more than about 50 □g/kg, no more than about 100 □g/kg, no more than about 500 □g/kg, no more than about 1 mg/kg, no more than about 5 mg/kg, no more than about 10 mg/kg, no more than about 25 mg/kg, no more than about 50 mg/kg, no more than about 100 mg/kg, no more than about 200 mg/kg, no more than about 300 mg/kg, no more than about 400 mg/kg, and no more than about 500 mg/kg of body weight of the subject. In one embodiment, the invention administers a dose which results in a concentration of the anti-YKL-40 antibody of the present invention of no more than about 1 pM, no more than about 10 pM, no more than about 100 pM, no more than about 1 nM, no more than about 10 nM, no more than about 100 nM, no more than about 1 μM, no more than about 2 μM, no more than about 3 μM, no more than about 4 μM, no more than about 5 μM, no more than about 6 μM, no more than about 7 μM, no more than about 8 μM, no more than about 9 μM and no more than about 10 μM in an individual. In another embodiment, the invention envisions administration of a dose which results in a concentration of the anti-YKL-40 antibody of the present invention between no more than about 1 pM, no more than about 10 pM, no more than about 100 pM, no more than about 1 nM, no more than about 10 nM, no more than about 100 nM, no more than about 1 μM, no more than about 2 μM, no more than about 3 μM, no more than about 4 μM, no more than about 5 μM, no more than about 6 μM, no more than about 7 μM, no more than about 8 μM, no more than about 9 μM and no more than about 10 μM in the plasma of an individual. Also contemplated are dosage ranges between any of the doses disclosed herein.

Typically, dosages which may be administered in a method of the invention to an animal, n some embodiments a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 μg to about 10 mg per kilogram of body weight of the animal. In some embodiments, the dosage will vary from about 3 μg to about 1 mg per kilogram of body weight of the animal.

The antibody may be administered to subject as frequently as several times daily, or it may be administered less frequently, such as once a day, twice a day, thrice a day, once a week, twice a week, thrice a week, once every two weeks, twice every two weeks, thrice every two weeks, once a month, twice a month, thrice a month, or even less frequently, such as once every several months or even once or a few times a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient (e.g., anti-YKL-40 antibody), the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. In some embodiments, the droplets provided by this route of administration have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and to may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Cells Producing Antibodies and Antigen Binding Fragments Thereof

In some embodiments, the invention is a cell or cell line (such as host cells) that produces at least one of the anti-YKL-40 antibodies, or antigen binding fragments, described herein. In one embodiment, the cell or cell line is a genetically modified cell that produces at least one of the anti-YKL-40 antibodies, or antigen binding fragments, described herein. In one embodiment, the cell or cell line is a hybridoma that produces at least one of the anti-YKL-40 antibodies, or antigen binding fragments, described herein.

Hybrid cells (hybridomas) are generally produced from mass fusions between murine splenocytes, which are highly enriched for B-lymphocytes, and myeloma “fusion partner cells” (Alberts et al., Molecular Biology of the Cell (Garland Publishing, Inc. 1994); Harlow et al., Antibodies. A Laboratory Manual (Cold Spring Harbor Laboratory, Cold Spring Harbor, 1988). The cells in the fusion are subsequently distributed into pools that can be analyzed for the production of antibodies with the desired specificity. Pools that test positive can be further subdivided until single cell clones are identified that produce antibodies of the desired specificity. Antibodies produced by such clones are referred to as monoclonal antibodies.

Also provided are nucleic acids encoding any of the antibodies, or antibody fragments, disclosed herein, as well as vectors comprising the nucleic acids. Thus, the antibodies and fragments of the invention can be generated by expressing the nucleic acid in a cell or a cell line, such as the cell lines typically used for expression of recombinant or humanized immunoglobulins. Thus, the antibodies and fragments of the invention can also be generated by cloning the nucleic acids into one or more expression vectors, and transforming the vector into a cell line such as the cell lines typically used for expression of recombinant or humanized immunoglobulins.

The genes encoding the heavy and light chains of immunoglobulins, or fragments thereof, can be engineered according to methods, including but not limited to, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al., 1992, J. Immunol. 148:1149). For example, genes encoding heavy and light chains, or fragments thereof, can be cloned from an antibody secreting cell's genomic DNA, or cDNA is produced by reverse transcription of the cell's RNA. Cloning is accomplished by conventional techniques including the use of PCR primers that hybridize to the sequences flanking or overlapping the genes, or segments of genes, to be cloned.

Nucleic acids encoding the antibody of the invention, or the heavy chain or light chain or fragments thereof, can be obtained and used in accordance with recombinant nucleic acid techniques for the production of the specific immunoglobulin, immunoglobulin chain, or a fragment or variant thereof, in a variety of host cells or in an in vitro translation system. For example, the antibody-encoding nucleic acids, or fragments thereof, can be placed into suitable prokaryotic or eukaryotic vectors, e.g., expression vectors, and introduced into a suitable host cell by an appropriate method, e.g., transformation, transfection, electroporation, infection, such that the nucleic acid is operably linked to one or more expression control elements, e.g., in the vector or integrated into the host cell genome.

In some embodiments, the heavy and light chains, or fragments thereof, can be assembled in two different expression vectors that can be used to co-transfect a recipient cell. In some embodiments, each vector can contain two or more selectable genes, one for selection in a bacterial system and one for selection in a eukaryotic system. These vectors allow for the production and amplification of the genes in a bacterial system, and subsequent co-transfection of eukaryotic cells and selection of the co-transfected cells. The selection procedure can be used to select for the expression of antibody nucleic acids introduced on two different DNA vectors into a eukaryotic cell.

Alternatively, the nucleic acids encoding the heavy and light chains, or fragments thereof, may be expressed from one vector. Although the light and heavy chains are coded for by separate genes, they can be joined, using recombinant methods. For example, the two polypeptides can be joined by a synthetic linker that enables them to be made as a single protein chain in which the V_{L} and V_{H} regions pair to form monovalent molecules (known as single chain Fv (scFv); see e.g., Bird et al., 1988, Science 242: 423-426; and Huston et al., 1988, Proc. Natl. Acad. Sci. USA 85:5879-5883).

The invention provides for an isolated nucleic acid molecule comprising a nucleic acid sequence encoding a heavy chain and/or a light chain, as well as fragments thereof. A nucleic acid molecule comprising sequences encoding both the light and heavy chain, or fragments thereof, can be engineered to contain a synthetic signal sequence for secretion of the antibody, or fragment, when produced in a cell. Furthermore, the nucleic acid molecule can contain specific DNA links which allow for the insertion of other antibody sequences and maintain the translational reading frame so to not alter the amino acids normally found in antibody sequences.

In accordance with the present invention, antibody-encoding nucleic acid sequences can be inserted into an appropriate expression vector. In various embodiments, the expression vector comprises the necessary elements for transcription and translation of the inserted antibody-encoding nucleic acid so as to generate recombinant DNA molecules that direct the expression of antibody sequences for the formation of an antibody, or a fragment thereof.

The antibody-encoding nucleic acids, or fragments thereof, can be subjected to various recombinant nucleic acid techniques known to those skilled in the art such as site-directed mutagenesis.

A variety of methods can be used to express nucleic acids in a cell. Nucleic acids can be cloned into a number of types of vectors. However, the present invention should not be construed to be limited to any particular vector. Instead, the present invention should be construed to encompass a wide variety of vectors which are readily available and/or known in the art. For example, the nucleic acid of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Viral vector technology is well known in the art and is described, for example, in Sambrook et al. (2012), and in Ausubel et al. (1999), and in other virology and molecular biology manuals. Viruses, which are useful as vectors include, but are not limited to, retroviruses, adenoviruses, adeno-associated viruses, herpes viruses, and lentiviruses. In some embodiments, a murine stem cell virus (MSCV) vector is used to express a desired nucleic acid. MSCV vectors have been demonstrated to efficiently express desired nucleic acids in cells. However, the invention should not be limited to only using a MSCV vector, rather any retroviral expression method is included in the invention. Other examples of viral vectors are those based upon Moloney Murine Leukemia Virus (MoMuLV) and HIV. In some embodiments, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193).

Additional regulatory elements, e.g., enhancers, can be used modulate the frequency of transcriptional initiation. A promoter may be one naturally associated with a gene or nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and fragments thereof.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter or cell-type specific promoter, which is a promoter that is active only in a desired tissue or cell. Tissue-specific promoters are well known in the art and include, but are not limited to, the HER-2 promoter and the PSA associated promoter sequences.

In order to assess the expression of the nucleic acids, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate nucleic acid and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing nucleic acids into a cell are known in the art. In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, laserporation and the like. Methods for producing cells comprising vectors and/or exogenous nucleic acids are well-known in the art. See, for example, Sambrook et al. (2012) and Ausubel et al. (1999).

Biological methods for introducing a nucleic acid of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a nucleic acid into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (e.g., an artificial membrane vesicle). The preparation and use of such systems is well known in the art.

Regardless of the method used to introduce exogenous nucleic acids into a host cell or otherwise expose a cell to the nucleic acid of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Kits

A kit is envisaged for every method described herein. The following description of a kit useful for diagnosing an inflammatory disorder or disease in a subject by measuring the level of a biomarker of the invention in a biological sample therefore is not intended to be limiting and should not be construed that way. In some embodiments, the biomarker is chitinase 3-like 1/Brp-39/YKL-40.

The kit may comprise a negative control containing a biomarker at a concentration of about the concentration of the biomarker which is present in a biological sample of an individual who does not have an inflammatory disorder or disease or does not have increased risk for an inflammatory disorder or disease. The kit may also include a positive control containing the biomarker at a concentration of about the concentration of the biomarker which is present in a biological sample of an individual who as an inflammatory disorder or disease or has increased risk for an inflammatory disorder or disease.

All the materials and reagents required for assaying chitinase 3-like 1/Brp-39/YKL-40 according to the present invention can be assembled together in a kit, such kit includes at least elements in aid of assessing the level of chitinase 3-like 1/Brp-39/YKL-40 in a biological sample obtained from an individual, and the instruction on how to do so.

A kit of the invention may have materials and reagents for detecting the chitinase 3-like 1/Brp-39/YKL-40 levels such as an immunoassay, or parts required to perform an immunoassay specific for chitinase 3-like 1/Brp-39/YKL-40 detection. Optionally, a kit may further or alternatively comprise elements for performing PCR based assays for the detection of chitinase 3-like 1/Brp-39/YKL-40 and determination of levels of to the same from biological samples. The kit of parts may further comprise equipment for obtaining one or more biological samples, such equipment may for example be syringes, vials or other. The components of the kit may also be provided in dried or lyophilized forms. When reagents or components are provided as a dried form, reconstitution generally is by the addition of a suitable solvent. It is envisioned that the solvent also may be provided in another container means.

The kit and components thereof may be packed for single use or for repeated usage, and the elements therein may be disposable such as to be disposed of after a single use or may be of a quality that allows repeated usage.

In addition to a diagnostic kit, the invention also provides a kit for use in therapeutic settings. In one embodiment, the kit comprises chitinase 3-like 1/Brp-39/YKL-40 or an activator thereof and an instructional material which describes, for instance, administering the chitinase 3-like 1/Brp-39/YKL-40 or an activator thereof to a subject as a prophylactic or therapeutic treatment or a non-treatment use as described elsewhere herein. In an embodiment, this kit further comprises a (preferably sterile) pharmaceutically acceptable carrier suitable for dissolving or suspending the therapeutic composition, comprising chitinase 3-like 1/Brp-39/YKL-40 or an activator thereof of the invention, for instance, prior to administering the molecule to a subject. Optionally, the kit comprises an applicator for administering the composition.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out embodiments of the present invention, and are not to be construed as limiting in any way the remainder of the disclosure.

### Example 1: Inhibition of Eosinophilic Inflammation in an Animal Model of Asthma

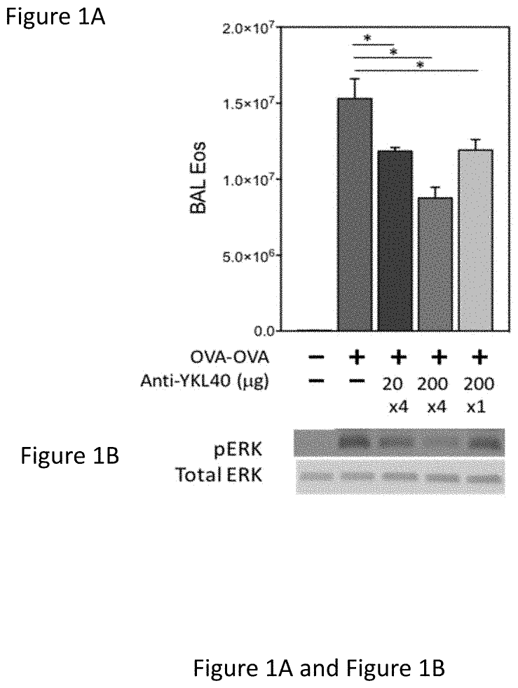

The effects of an anti-YKL-40 antibody were assessed in a murine model of asthma to assess effects on inflammation and ERK activation. BRP-39 knockout/YKL-40 transgenic animals were sensitized with ovalbumin (OVA) by intraperitoneal injection for 2 weeks and then challenged with OVA by ultrasonic nebulization using standard protocols. Animals were treated with anti-YKL-40 antibody prior to nebulization every other day four times at 200 μg/dose, 20 μg/dose, or were treated 200 μg once followed by sacrifice and standard bronchoalveolar lung lavage (FIG. 1). As can be seen in the FIG. 1, untreated animals had significant eosinophilic inflammation that was blocked by anti-YKL-40 in a dose response fashion at doses at or below the <20 mg/kg selection criterion (˜8 mg/kg and 20 mg/kg per week). Antibody-treated mice also had lower numbers of total inflammatory cells compared to control IgG treated mice. In addition to inhibiting inflammation, downstream pharmacodynamic effects of anti-YKL-40 have been evaluated by examining ERK phosphorylation in lung lysates from OVA treated mice. This analysis showed reduced ERK phosphorylation in a dose response fashion compared to control that is approximately 70% of YKL-40 dependent effect, again meeting the selection criterion (FIG. 1).

### Example 2: Anti-Cancer Effects of Anti-YKL-40 Antibody

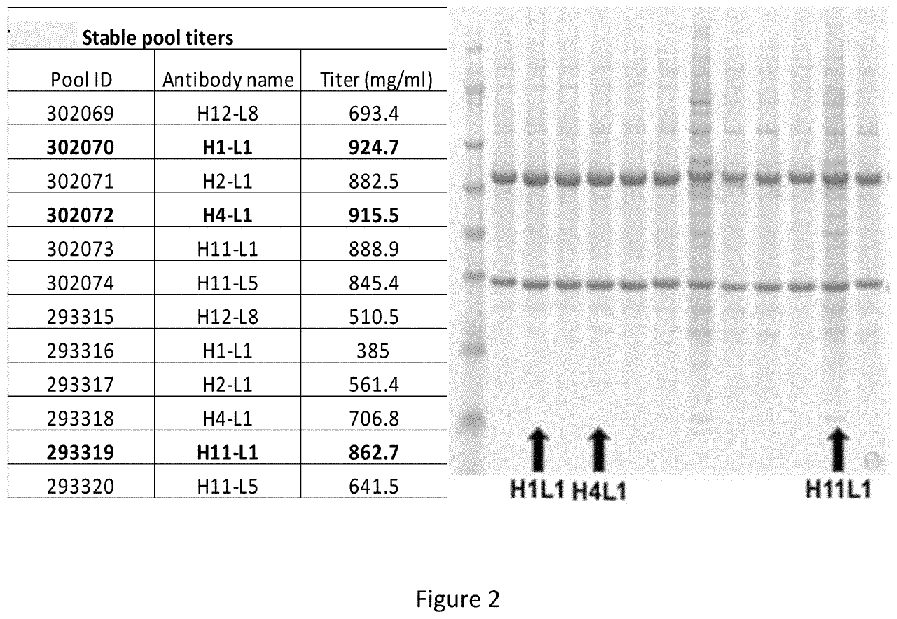

The effects of anti-YKL-40 mAbs in cancer models including a primary lung cancer model and a melanoma metastasis model have been evaluated. In the lung cancer model, an oncogene is turned on and a tumor suppressor gene is turned-off. This induces a primary lung cancer to develop in the mouse lung (mass is in the lung in the upper left panel in FIG. 2). As can be seen in FIG. 2 under “anti-YKL-40” on the left, treatment with antibody, significantly reduced tumor growth. Second, a melanoma metastasis model was assessed. Animals were injected by tail vein with murine melanoma cells at different doses and were pretreated with control IgG or anti-YKL-40 antibody. As can be seen on the right side of FIG. 2, animals treated with anti-YKL-40 had near complete absence of melanoma metastases on the surface of the lungs, compared to control IgG treated mice. Taken together, these studies demonstrate anti-YKL-40 antibody has significant in vivo to modulatory effects on allergic inflammation and ERK phosphorylation in a dose response fashion that meets dosing selection criteria against both murine BRP-39 and YKL-40 mediated models of disease.

### Example 3: Inhibition of Cell Proliferation by Anti-YKL-40 Antibody

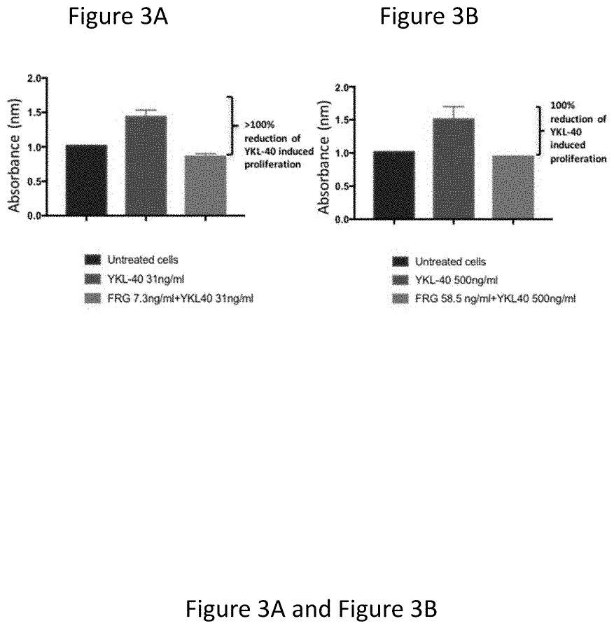

The effects of anti-YKL-40 on proliferation in U937 cells that were generated using CRISPR/CAS9 gene editing system to knockout YKL-40 was also assessed. These cells were specifically generated for this purpose to eliminate endogenous YKL-40 interference (FIG. 3). In these studies, U937 YKL-40 KO cells were plated at day 0 at 10000 cells/well in flat bottom 96 wells plates. YKL-40 and anti-YKL-40 mAb were premixed on ice for 30 minutes. Cells were stimulated at day 1 for 72 hours with YKL-40 31 ng/ml and 500 ng/ml and a combination of YKL-40 and anti-YKL-40 mAb FRG. Each condition was assessed in duplicate wells. Assay was performed by adding 20 μl of the CellTiter 96® Aqueous One Solution Reagent directly to culture wells, incubating for 3 hours and then recording the absorbance at 490 nm with a 96-well plate reader. As can be seen in FIG. 3, antibody blocks YKL-40 induced proliferation in a dose-dependent fashion.

### Example 4: Binding Affinity of Anti-YLK-40 mAb to rhYKL-40 Proteins

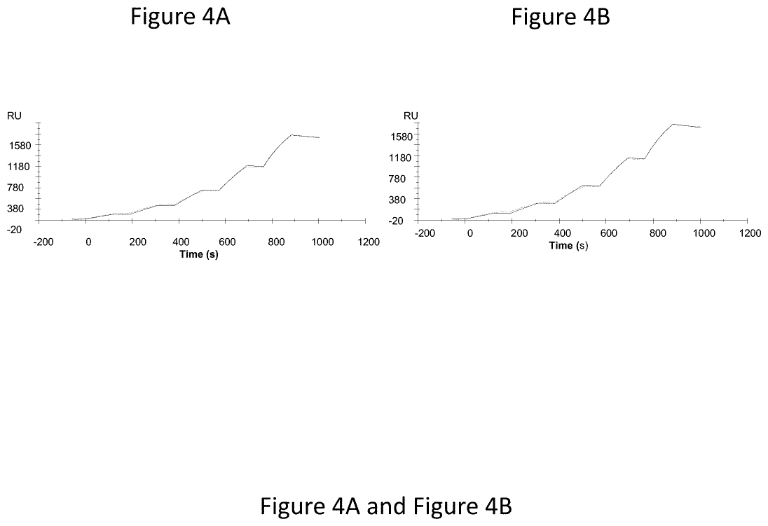

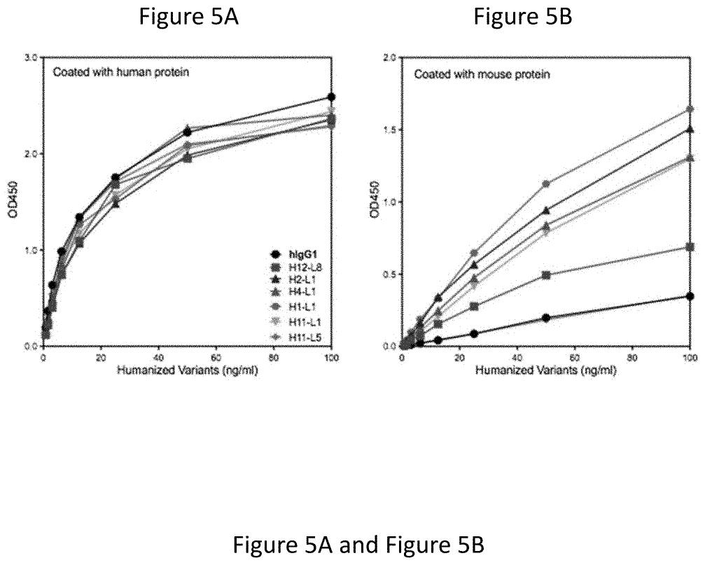

The affinity of FRG to 2 different recombinant human YKL-40 proteins was assessed on a Biocore system. Rh YKL-40 proteins were generated using two different mammalian expression systems or were purchased from R&D. These proteins were immobilized on the CM5 chip via direct amine coupling at the level of a few thousand RUs on cell 2 and 3 of the chip. In addition, for a negative control, IL-13 protein was immobilized by amine-coupling at high density levels on cell 4 of the same CM5 chip (FIG. 4). Antibody was then floated over the chip and binding measured. As seen in FIG. 5, antibody bound in a nearly identical fashion to both YKL-40 proteins. For both proteins, the affinity was determined as ˜5 nM (0.000287 kd) for both selected rhYKL-40 proteins. No binding was observed between Ab and a negative control (IL-13 protein). There was no binding observed between YLK-40 (from either source) and control IgG. These studies demonstrate that antibody has strong affinity for YKL-40.

Western blot analyses using both denaturing and non-denaturing conditions was performed and confirmed that the anti-YKL-40 Ab binds to human, murine and rat YKL-40 at similar affinities (data not shown). Most importantly, the antibody has higher affinity under non-denaturing conditions to both murine at human YKL-40. This indicates it binds better to native configured YKL-40. Based on these experiments and the titration ELISA shown in FIG. 4 with humanized mAb, affinity of mAb for murine BRP-39 was estimated to be about half its affinity to human YKL-40. Interestingly, as seen in FIG. 5, humanized Ab has improved affinity for murine BRP-39 compared to the murine mAb.

### Example 5: Assessment of Anti-YKL-40 Antibody Stable Pools

96 humanized antibody variants were generated and assessed for transient growth titers, binding affinity, and species cross-reactivity to murine, primate, and human homologues of CHI3L1/YKL-40. Based on these assessments, six of the 96, were selected for additional study (i.e., H1-L1, H4-L1, H11-L1, H2-L1, H11-L5, H12-L8).

To generate stable pools, genes for the heavy and light chains of the 6 candidates were codon optimized and cloned into ATUM's dual promoter, stable expression constructs. Expression constructs capable of two different stringencies of selection were constructed, resulting in 2 constructs for each candidate antibody, or 12 constructs. These are listed in FIG. 2. Expression constructs were co-transfected at different titers with LPN transposase mRNA into Horizon HD-BIOP3 CHO-GS KO cells. Stable pools were selected in the absence of glutamine. The cultures were assessed for productivity and cell growth on day 5, 7, 9, 11 and 14. Recovered stable pools were analyzed for productivity in 10 ml Tubespins under non-optimized fed batch conditions (productivity titers are shown in FIG. 2 in mg/liter). Clarified harvest was collected on Day 14 and analyzed by protein A binding on an Octet for antibody titer, as well as by reducing SDS-PAGE for heavy chain and light chain expression (FIG. 2). Based on these results, the highest expressing pools of antibodies H1-L1, H4-L1, and H11-L1 were selected for further testing

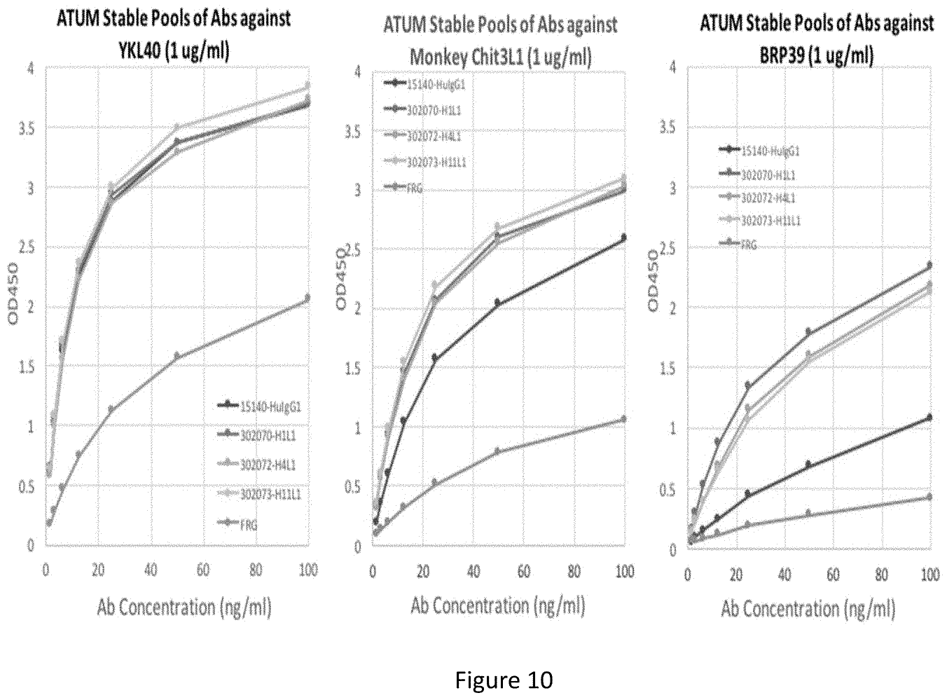

Next, binding affinity and species cross-reactivity were tested on the 3 humanized stable pools by titration ELISA using human and primate CHI3L1/YKL-40 and the murine homologue, BRP-39 (FIG. 10). As can be seen in the figure, all three clones show higher affinity to all three species of CHI3L1/YKL40/BRP-39 compared to the original murine version of the antibody (FRG, in blue). As expected, all the pools showed the highest affinity to human CHI3L1/YKL-40, followed by primate, and murine protein in a range adequate to conduct toxicity and PK/PD studies in mice and primates (within 2-fold among all species examined). Importantly and consistent with other studies, H1-L1 had the strongest affinity for murine BRP-39. In addition, gel electrophoresis and western blotting under denaturing and non-denaturing conditions was conducted to confirm binding to the target, CHI3L1/YKL40. Recombinant murine, human and primate (Cynomolgus monkey) protein were pretreated with b-mercaptoethanol (Reduced) or without (Non-reduced) sample buffer. 500 ng/lane of pretreated recombinant protein was loaded onto SDS-PAGE gel or Native (non-reducing) PAGE gel. After electrophoresis and western blotting, using the 3 humanized pools for detection: H1-L1, H4-L1, and H1-1L1 (not shown), and the original mAb, FRG. Goat anti-human IgG-HRP was used for detecting H1-L1 and H4-L1, and H11-L1, and mouse-IgG-k-Binding Protein-HRP for FRG at 1:4,000. Evident FIG. 10 is that both H1-L1 and H4-L1 have similar ability to detect denatured and non-denatured CHI3L1. Importantly, while the detection of murine homologue (BRP-39) is significantly less in this assay, H1-L1 appears to have slightly higher affinity for non-denatured murine protein compared to H4-L1. Therefore, by both titration ELISA and Western blot, H1-L1 demonstrated the strongest affinity to all three species of CHI3L1/YKL-40.

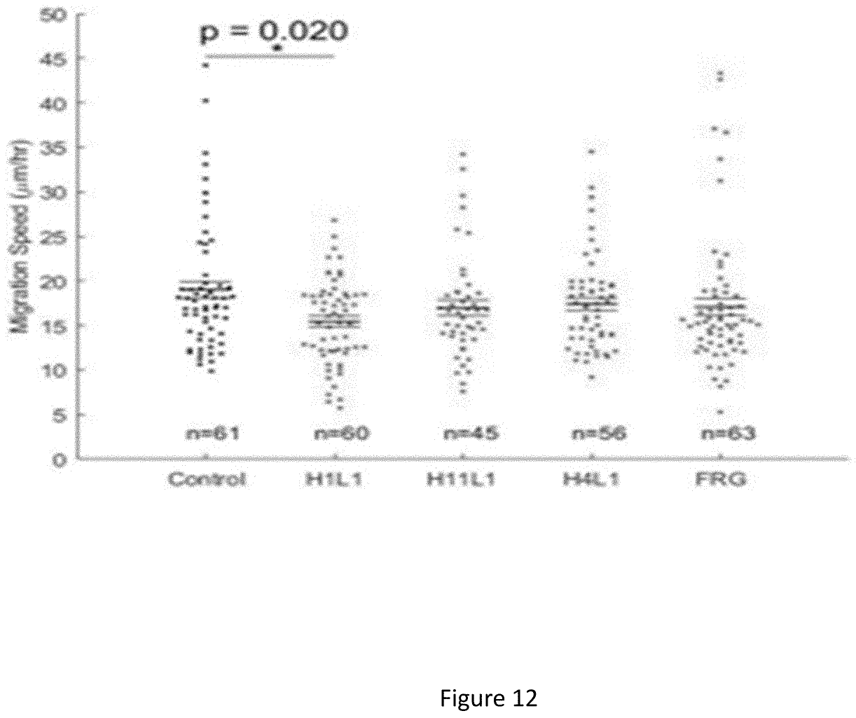

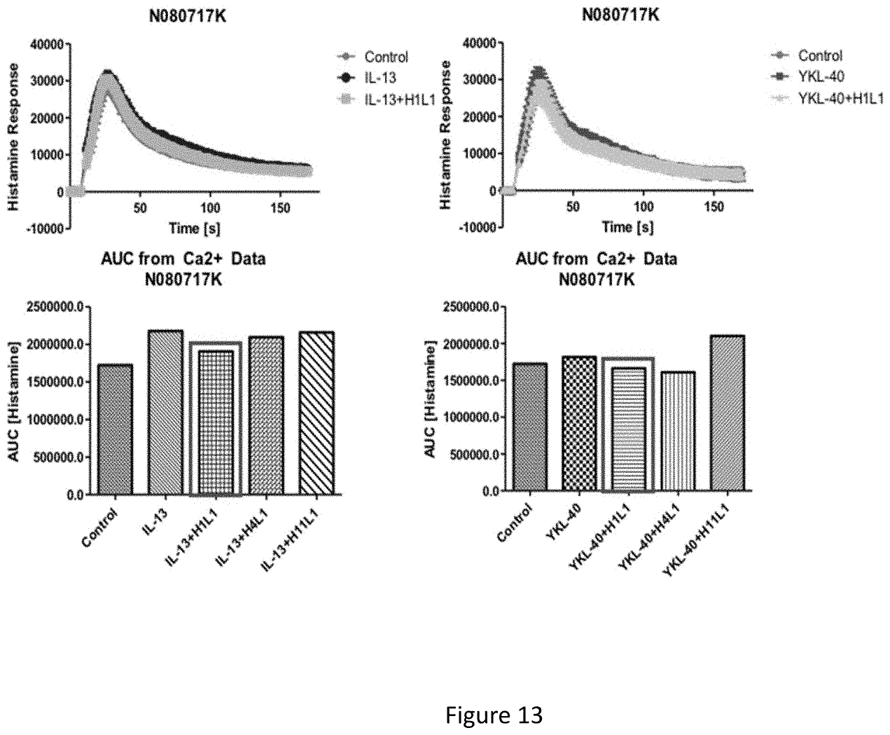

Lastly, the inhibitory effects of the three humanized antibodies (H1-L1, H4-L1, and H1-1L1) were evaluated to ensure that the antibody with the greatest inhibitory capacity is selected for future studies. This was done using a high throughput assay where the migration trajectories of human melanoma cells that respond to YKL-40 are tracked for 500 minutes with the speed measured every 10 minutes (FIG. 12). Cells are treated with H1-L1, H4-L1, and H1-111, and FRG. Conditions are shown on the axis legend and each dot represents the average speed of an individual cell and lines represent mean and standard error. As can be seen in the FIG. 12, compared to control, H1L1 showed the greatest inhibitory effect on cell migration speed compared to control. Lastly, the ability of H1-L1, H4-L1, and H1-1L1 to inhibit human airway smooth muscle activation and bronchoconstriction in precision cut human lung slices (PCLS) (FIG. 13) was compared. Human airway smooth muscle cells were treated with YKL-40 which augmented histamine induced calcium flux. Anti-YKL-40 H1-L1 and H4-L1 antibodies decreased the peak calcium flux and AUC. When cells were stimulated with IL-13, there is augmentation of peak calcium flux that is inhibited by H1-L1. Evaluation primary human airway smooth muscle cells from a patient with asthma by Western blots showed that the addition of YKL-40 at 10 ng/ml increased histamine-induced phosphorylation of both MLC and MYPT1 which was inhibited by H1-L1 and PCLS studies demonstrated H1-L1 augments bronchodilation.

Evaluating all criteria including species cross-reactivity, binding affinity, productivity, and bioactivity, H1L1 exhibited superior results compared to H4L1 and H11L1. A research cell bank has been established for H1-L1 and over 6.0 grams of non-GLP research grade material has been produced.

Production of the anti-YKL-40 H1-L1 stable pool, generating 6.7 grams of humanized antibody from 5 liters of bioreactor serum free culture has been completed. The product was over 95% monomer with less than 5% aggregation, so no polishing was needed following purification. In addition, the H1-L1 is endotoxin free and the final concentration in PBS is 4.87 mg/ml. The antibody demonstrates excellent stability with no deterioration after freeze-thaw 5 times. The 6.7 grams of antibody is stored-in 50 ml aliquots.

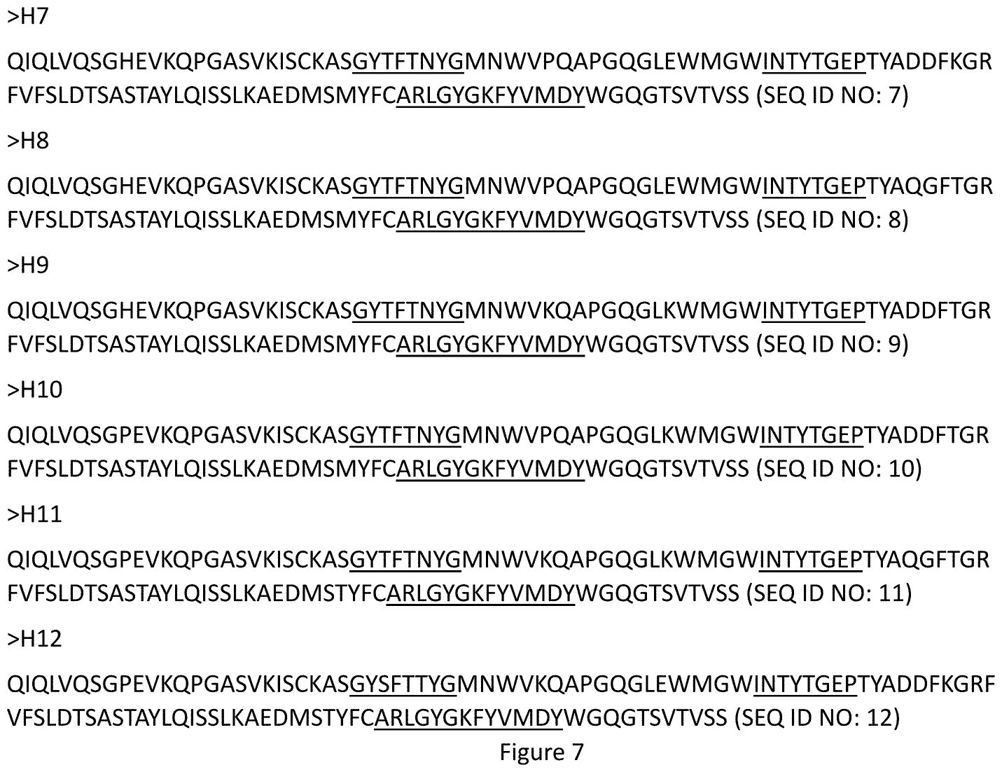

An antibody that binds specifically to the antigen binding domain of H1L1-termed an “anti-ID” antibody reagent, E6, has been developed. This reagent is used for detection of H1-L1 in pharmacokinetic serum samples from animals and humans systemically administered anti-YKL-40 in clinical trials. This information is used to determine the doses of anti-YKL-40 to be used in preliminary toxicity studies, formal GLP-toxicity studies, and Phase I, II, and III trials once IND is granted. The anti-ID antibody needs to be specific against the complimentary-determining regions (CDRs) of the anti-YKL-40 H1-L1 and do not cross-react with non-specific human antibodies that are present in the serum. The development of an anti-ID antibody can take as long as developing a lead candidate biologic itself. An anti-ID antibody against H1L1 was generated (FIG. 7). Phage display was employed to screen for specific binding to the target sequence. From this process 3 clones were identified of which the E6 scFv clone showed the highest specificity and strongest affinity to both native and denatured H1-L1. This clone also binds to the two anti-YKL-40 antibodies, H4-L1 and H11-L1. Evident in FIG. 7 is that the E6 scFv demonstrates 2-5 fold binding over background scFv and IgG1 control and is capable of binding to both native and denatured (1% SDS+/−DTT) H1-L1, so specific assay characteristics are flexible and can be adjusted depending on species and tissue compartment being examined. The assay uses a sandwich MSD assay (electrochemiluminescence) format that will use an anti-IgG 1 capture antibody and the E6 clone (either in an IgG1 format or the myc tagged scFv) for detection. This will provide the highest specificity and sensitivity (lower limit of detection<50 pg/ml). Spike-in experiments with human serum from asthmatics with varying YKL-40 levels are being conducted to optimize assay characteristics required to measure total H1-L1 or H1-L1 bound to YKL-40 or its mouse and primate homologue in the serum and/or plasma.

### Example 6: Non-GLP Toxicity PK Study in Primates

Using non-GLP material produced above, single dose toxicity studies will be initiated in mice and primates. The industry standard in developing biologic therapeutic antibodies is to conduct preliminary toxicity studies in mice and primates to demonstrate safety and generate PK data for the selection of doses to be used in toxicity studies. These studies are conducted using investigational product produced under GMP and required for IND submission. With over 6.0 grams of H1-L1 antibody in hand, there is more than enough investigational product available to pursue preliminary toxicity studies in mice and primates. The mouse studies are in process using a range of H1L1 doses.

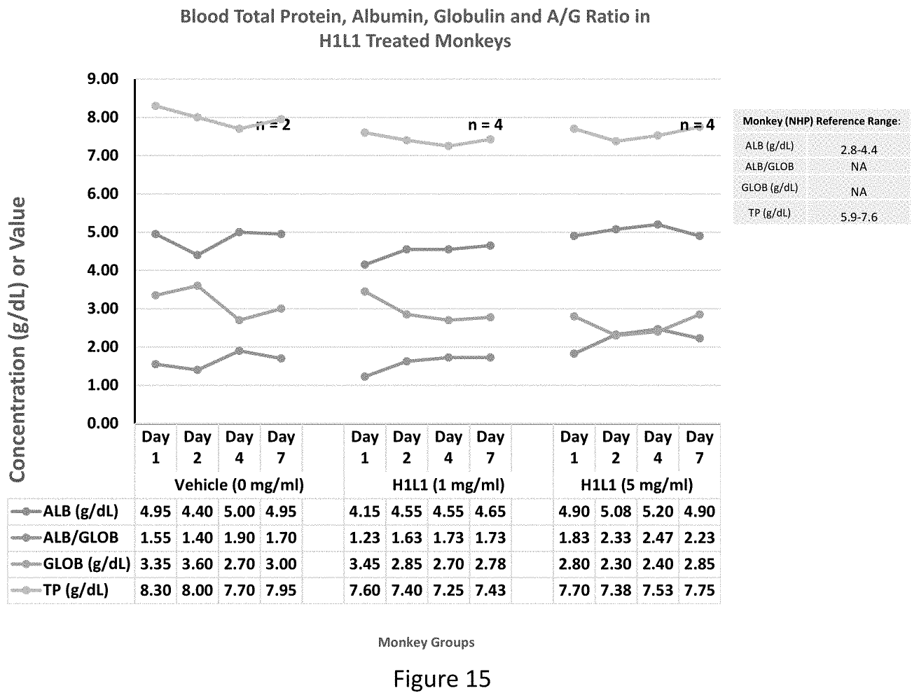

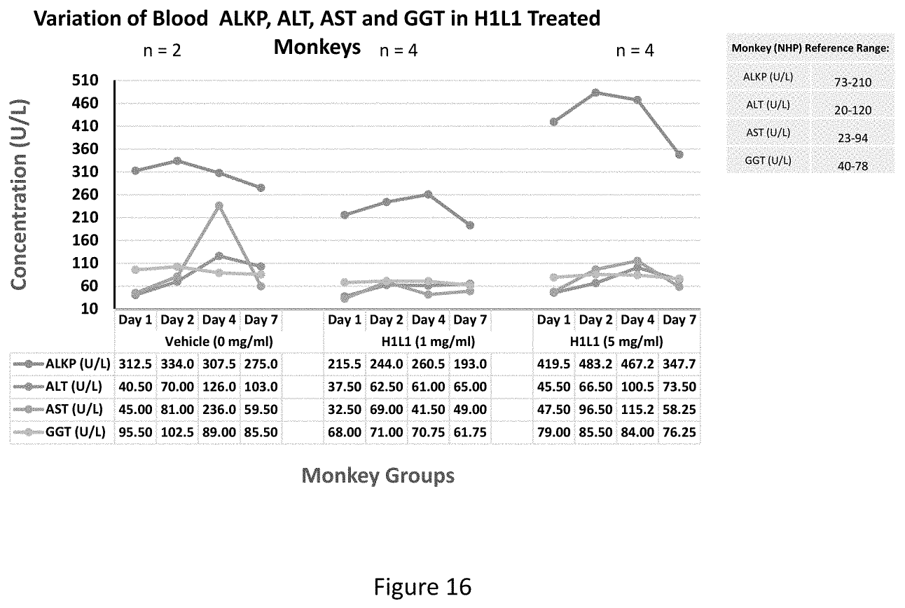

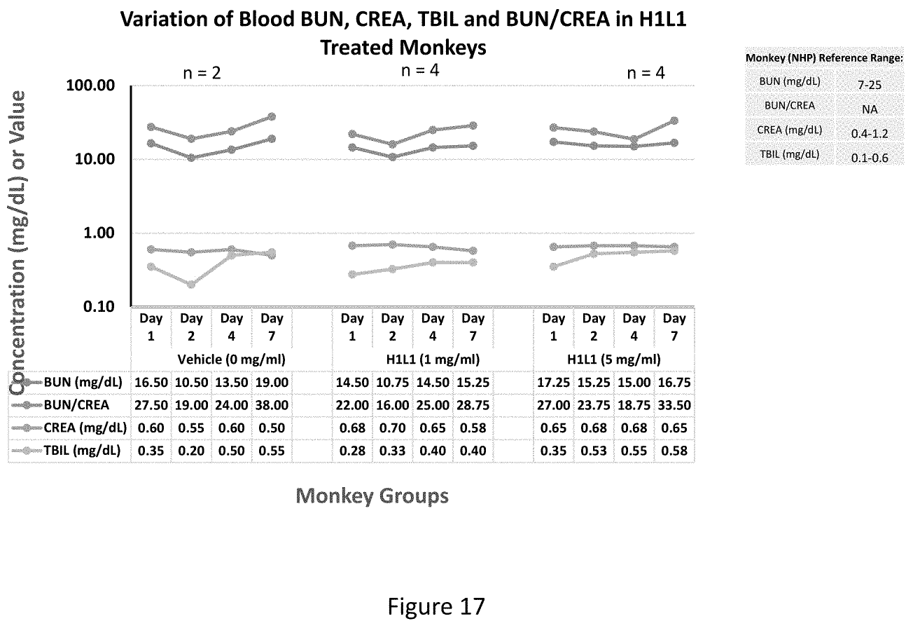

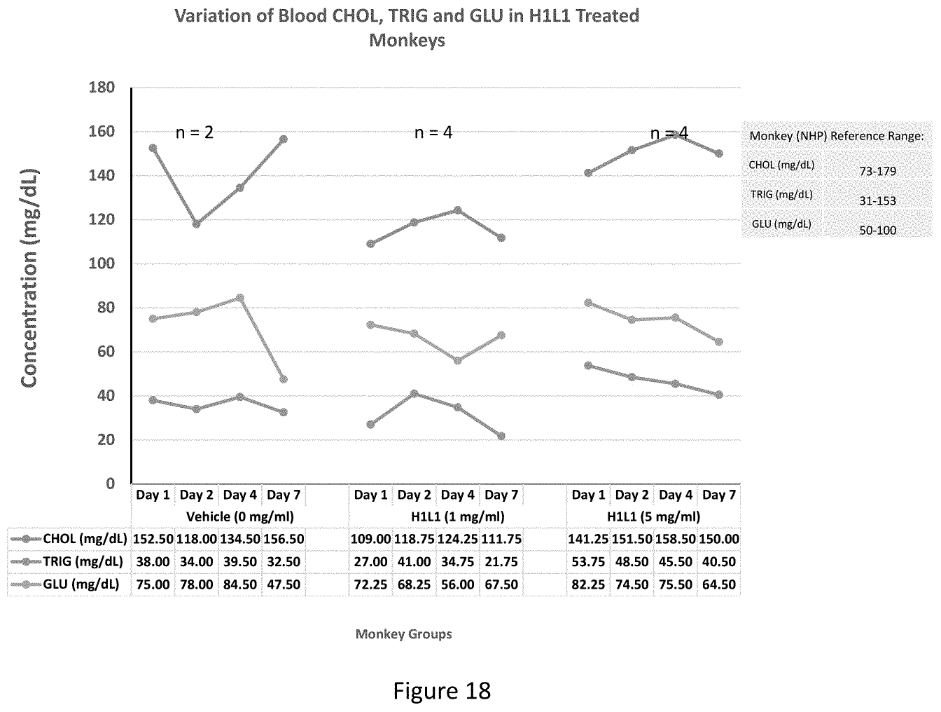

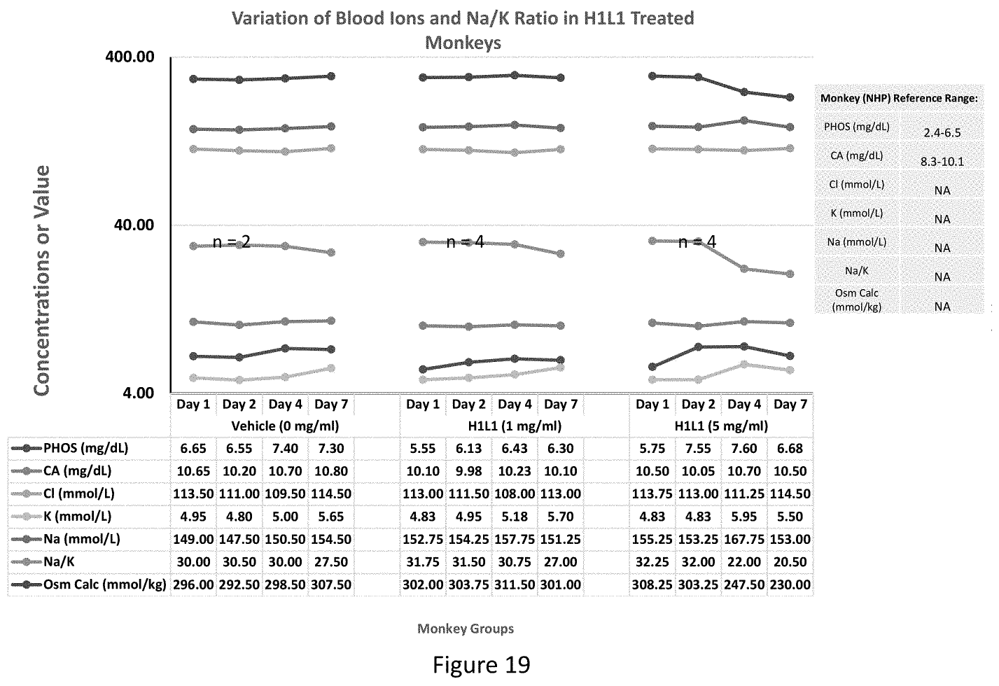

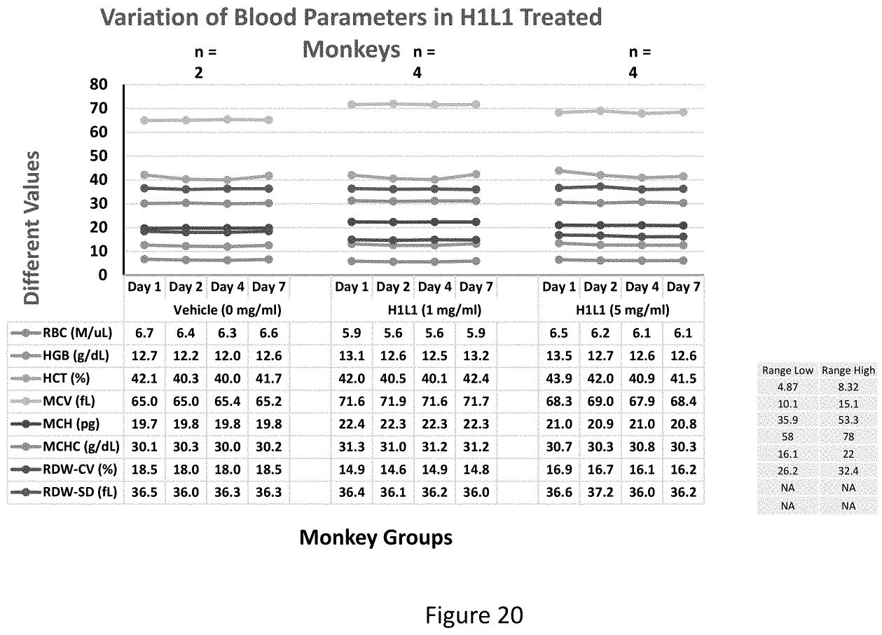

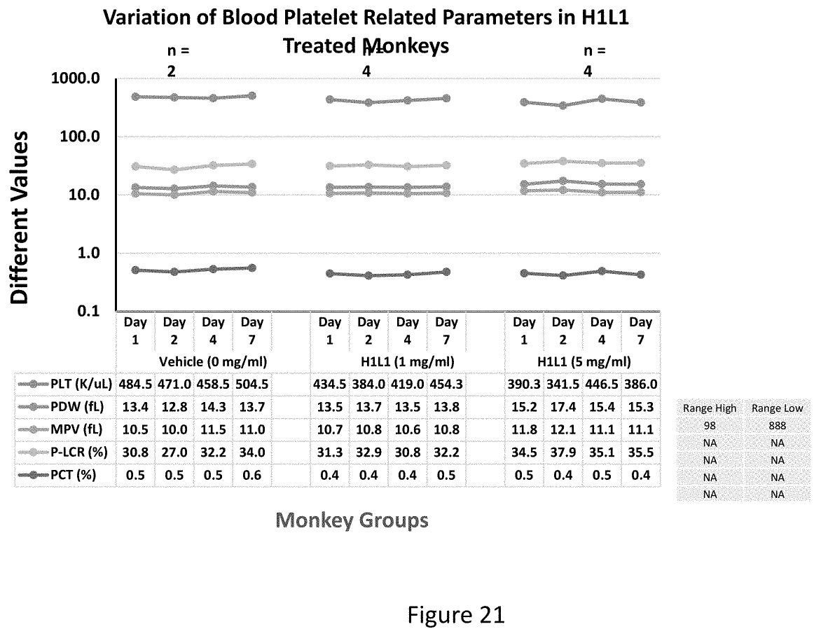

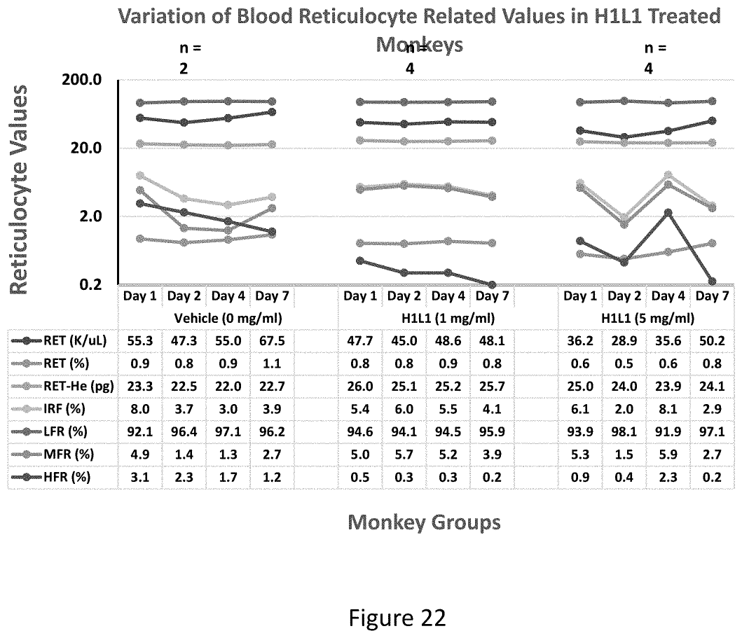

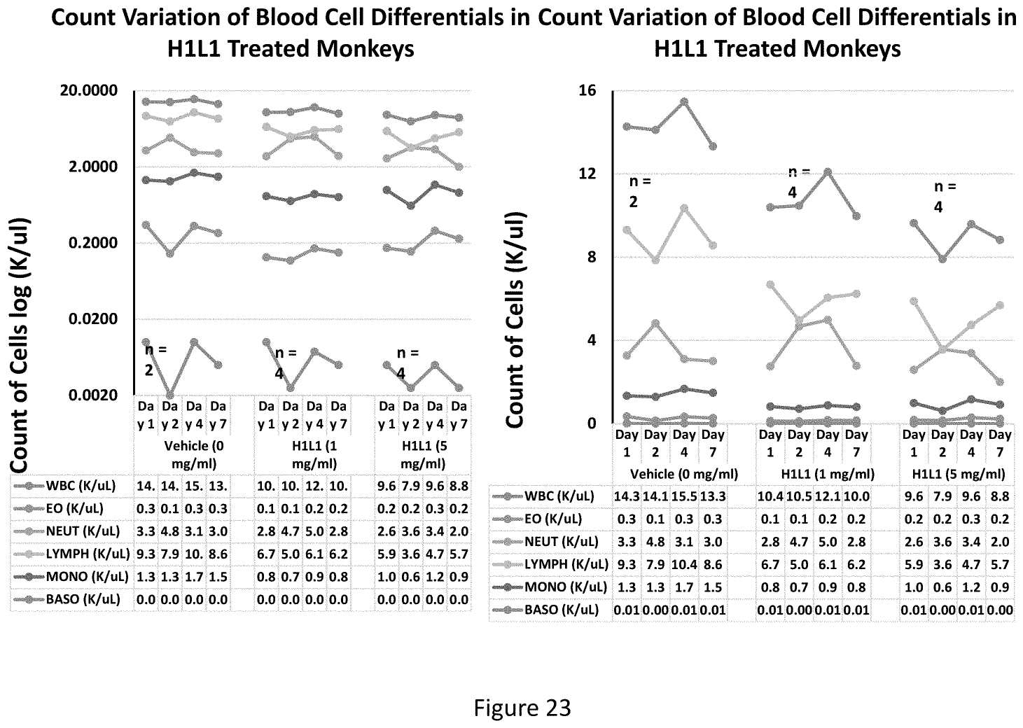

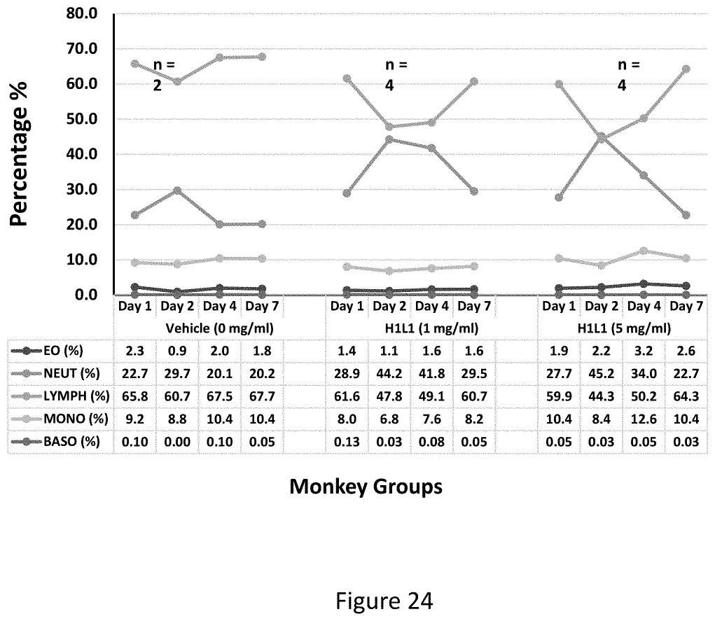

To demonstrate that H1-L1 shows no overt toxicity in vivo, a single dose observation study is being conducted in Cynomolgous monkeys. The animals were blindly dosed with vehicle or two doses of H1-L1. The control group (vehicle alone) had one male and one female monkey, and the two H1-L1 dosing groups had 2 males and 2 females to ensure that any potential toxicity that could be related to sex is evaluated. The drug was delivered IV over one hour Animals will be observed for food consumption daily and weighed at the beginning and end of a one-week observation period. Clinical signs of toxicity will be monitored on a daily basis and as needed. Serum and plasma will be collected at time zero and 0, 1, 6, 24, 48, 72, 168 hours for PK measurements of H1-L1 using the anti-ID assay outlined above. Complete blood counts, serum chemistries will be done daily for 1 week. However, if any toxicity is evident, the cause will be determined, and modifications made as needed to mitigate risk. The results of blood counts and serum chemistries is shown in FIGS. 15-24. The abbreviations used in FIGS. 15-24 are as follows: ALB=albumin;
ALKP=alkaline phosphatase;
ALT (SGPT)=alanine aminotransferase;
AST=aspartate aminotransferase;
BUN (UREA)=blood urea nitrogen;
CA=calcium;
CHOL=cholesterol;
CREA=creatinine;
GGT=gamma-glutamyltransferase;
GLU=glucose;
PHOS=phosphorous;
TBIL=total bilirubin;
TP=total protein;
TRIG=triglycerides;
ALB/GLOB=albunin/globulin ratio;
GLOB=globulin;
Cl=Chloride;
K=Potassium;
Na=Sodium
RBC=red blood cells;
HGB=hemoglobin;
HCT=hematocrit;
MCV=mean corpuscular volume;
MCH=mean corpuscular hemoglobin;
MCHC=mean corpuscular hemoglobin concentration;
RDW-CV=red cell distribution width-coefficient of variation;
RDW-SD=red cell distribution width-standard deviation;
PLT=platelets;
PDW=platelet distribution width;
MPV=mean platelet volume;
P-LCR=platelet larger cell ratio;
PCT=plateletcrit;
RET=reticulocytes;
IRF=immature reticulocyte fraction;
LFR=low fluorescence reticulocytes;
MFR=middle fluorescence reticulocytes;
HFR=high fluorescence reticulocytes;
WBC=white blood cells;
EO=eosinophil;
NEUT=neutrophils;
LYMPH=lymphocytes;
MONO=monocytes;
BASO=basophil;
M/uL=millions per cubic millimeter;
K/uL=thousands per cubic milliliter;
g/dL=grams per deciliter;
fL=femtoliters.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An antibody that specifically binds to human YKL-40,
wherein the antibody comprises
one of the vHs selected from the group consisting of: VH-V1 SEQ ID NO:1; VH-V2: SEQ ID NO:2; VH-V3: SEQ ID NO:3; VH-V4: SEQ ID NO:4; VH-V5: SEQ ID NO:5; VH-V6: SEQ ID NO: 6; VH-V7: SEQ ID NO: 7; VH-V8: SEQ ID NO: 8; VH-V9: SEQ ID NO: 9; VH-V10: SEQ ID NO: 10; VH-V11: SEQ ID NO: 11; and VH-V12: SEQ ID NO: 12, and
one of the vLs selected from the group consisting of: VK-V1 SEQ ID NO:13; VK-V2: SEQ ID NO:14; VK-V3: SEQ ID NO:15; VK-V4: SEQ ID NO:16; VK-V5: SEQ ID NO:17; VK-V6: SEQ ID NO:18; VK-V7: SEQ ID NO:19; and VK-V8: SEQ ID NO:20.

2. The antibody of claim 1, where is the vH is SEQ ID NO:1 and the vL is SEQ ID NO:13.

3. The antibody of claim 1, where is the vH is SEQ ID NO:4 and the vL is SEQ ID NO:13.

4. The antibody of claim 1, where is the vH is SEQ ID NO:11 and the vL is SEQ ID NO:13.

5. The antibody of claim 1, where is the vH is SEQ ID NO:2 and the vL is SEQ ID NO:13.

6. The antibody of claim 1, where is the vH is SEQ ID NO:11 and the vL is SEQ ID NO:17.

7. The antibody of claim 1, where is the vH is SEQ ID NO:12 and the vL is SEQ ID NO:20.

8. A method of treating an inflammatory disease or disorder in an individual, comprising the step of administering to said individual the antibody of claim 1.

9. The method of claim 12, wherein the disease or disorder is at least one selected from the group consisting of: asthma, cancer, lung cancer, lung disease, and metastatic melanoma.

10. The method of claim 12, wherein administration of the antibody inhibits the activation of eosinophils.

11. A method of reducing the inflammation in an individual, comprising the step of administering to said individual the antibody of claim 1.

12. The method of claim 15, wherein the individual has a disease or disorder selected from the group consisting of: asthma, cancer, lung cancer, lung disease, and metastatic melanoma.

13. A method of treating cancer in an individual, comprising the step of administering to said individual the antibody of claim 1.

14. The method of claim 17, wherein the cancer is lung cancer, melanoma, or metastatic melanoma.


SEQUENCE LISTING (29 sequences)

SEQ ID NO: 1 - protein, 120 aa, Artificial Sequence
  humanized
QIQLVQSGSELKKPGASVKISCKASGYTFTNYGMNWVRQAPGQGLEWMGWINTYTGEPTYADDFKGRFVFSLDTSVSTAYLQISSLKAEDTSVYFCARLGYGKFYVMDYWGQGTSVTVSS

SEQ ID NO: 2 - protein, 120 aa, Artificial Sequence
  humanized
QIQLVQSGSELKKPGASVKISCKASGYTFTNYGMNWVRQAPGQGLEWMGWINTYTGEPTYAQGFTGRFVFSLDTSVSTAYLQISSLKAEDTSVYFCARLGYGKFYVMDYWGQGTSVTVSS

SEQ ID NO: 3 - protein, 120 aa, Artificial Sequence
  humanized
QIQLVQSGPELKKPGASVKISCKASGYTFTNYGMNWVRQAPGQGLKWMGWINTYTGEPTYADDFTGRFVFSLDTSVSTAYLQISSLKAEDTSVYFCARLGYGKFYVMDYWGQGTSVTVSS

SEQ ID NO: 4 - protein, 120 aa, Artificial Sequence
  humanized
QIQLVQSGSELKKPGASVKISCKASGYTFTNYGMNWVRQAPGQGLKWMGWINTYTGEPTYADDFKGRFVFSLDTSVSTAYLQISSLKAEDTSVYFCARLGYGKFYVMDYWGQGTSVTVSS

SEQ ID NO: 5 - protein, 120 aa, Artificial Sequence
  humanized
QIQLVQSGPELKKPGASVKISCKASGYTFTNYGMNWVKQAPGQGLKWMGWINTYTGEPTYAQGFTGRFVFSLDTSVSTAYLQISSLKAEDTSTYFCARLGYGKFYVMDYWGQGTSVTVSS

SEQ ID NO: 6 - protein, 120 aa, Artificial Sequence
  humanized
QIQLVQSGPELKKPGASVKISCKASGYTFTSYAMNWVKQAPGQGLKWMGWINTYTGEPTYADDFKGRFVFSLDTSVSTAYLQISSLKAEDTSVYFCARLGYGKFYVMDYWGQGTSVTVSS

SEQ ID NO: 7 - protein, 120 aa, Artificial Sequence
  humanized
QIQLVQSGHEVKQPGASVKISCKASGYTFTNYGMNWVPQAPGQGLEWMGWINTYTGEPTYADDFKGRFVFSLDTSASTAYLQISSLKAEDMSMYFCARLGYGKFYVMDYWGQGTSVTVSS

SEQ ID NO: 8 - protein, 120 aa, Artificial Sequence
  humanized
QIQLVQSGHEVKQPGASVKISCKASGYTFTNYGMNWVPQAPGQGLEWMGWINTYTGEPTYAQGFTGRFVFSLDTSASTAYLQISSLKAEDMSMYFCARLGYGKFYVMDYWGQGTSVTVSS

SEQ ID NO: 9 - protein, 120 aa, Artificial Sequence
  humanized
QIQLVQSGHEVKQPGASVKISCKASGYTFTNYGMNWVKQAPGQGLKWMGWINTYTGEPTYADDFTGRFVFSLDTSASTAYLQISSLKAEDMSMYFCARLGYGKFYVMDYWGQGTSVTVSS

SEQ ID NO: 10 - protein, 120 aa, Artificial Sequence
  humanized
QIQLVQSGPEVKQPGASVKISCKASGYTFTNYGMNWVPQAPGQGLKWMGWINTYTGEPTYADDFTGRFVFSLDTSASTAYLQISSLKAEDMSMYFCARLGYGKFYVMDYWGQGTSVTVSS

SEQ ID NO: 11 - protein, 120 aa, Artificial Sequence
  humanized
QIQLVQSGPEVKQPGASVKISCKASGYTFTNYGMNWVKQAPGQGLKWMGWINTYTGEPTYAQGFTGRFVFSLDTSASTAYLQISSLKAEDMSTYFCARLGYGKFYVMDYWGQGTSVTVSS

SEQ ID NO: 12 - protein, 120 aa, Artificial Sequence
  humanized
QIQLVQSGPEVKQPGASVKISCKASGYSFTTYGMNWVKQAPGQGLEWMGWINTYTGEPTYADDFKGRFVFSLDTSASTAYLQISSLKAEDMSTYFCARLGYGKFYVMDYWGQGTSVTVSS

SEQ ID NO: 13 - protein, 112 aa, Artificial Sequence
  humanized
DVVMTQSPLSLPVTLGQPASISCRSSQSLVHSNGNTYLNWYQQRPGQSPRLLIYKVSNRFSGVPDRFSGSGSGTDFTLKISRVEAEDVGVYFCSQSTHVTWTFGGGTKLEIK

SEQ ID NO: 14 - protein, 112 aa, Artificial Sequence
  humanized
DVVMTQSPLSLPVTLGQPASISCRSSQSLVHSDGNTYLHWYQQRPGQSPRLLIYKVSNRFSGVPDRFSGSGSGTDFTLKISRVEAEDVGVYFCSQSTHVTWTFGGGTKLEIK

SEQ ID NO: 15 - protein, 112 aa, Artificial Sequence
  humanized
DVVMTQSPLSLPVTLGQPASISCRSSQSLVHSNGNTYLHWYQQRPGQSPRLLIYKVSNRDSGVPDRFSGSGSGTDFTLKISRVEAEDVGVYFCSQSTHVTWTFGGGTKLEIK

SEQ ID NO: 16 - protein, 112 aa, Artificial Sequence
  humanized
DVVMTQSPLSLPVTLGQPASISCRSSQSLVHSNGNTYLHWFQQRPGQSPRLLIYKVSNRFSGVPDRFSGSGSGTDFTLKISRVEAEDVGVYFCSQSTHVTWTFGGGTKLEIK

SEQ ID NO: 17 - protein, 112 aa, Artificial Sequence
  humanized
DIVMTQTPLSLSVTPGQPASISCKSSQSLVHSNGNTYLHWYLQKPGQSPQLLIYKVSNRFSGVPDRFSGSGSGTDFTLKISRVEAEDVGVYYCSQSTHVTWTFGGGTKLEIK

SEQ ID NO: 18 - protein, 112 aa, Artificial Sequence
  humanized
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVTWTFGGGTKLEIK

SEQ ID NO: 19 - protein, 112 aa, Artificial Sequence
  humanized
DVVMTQSPLSLPVTLGQPASISCRSSQSLVHSNGNTYLHWFLQRPGQSPRLLIYKVSNRFSGVPDRFSGSGSGTDFTLKISRVEAEDVGVYYCSQSTHVTWTFGGGTKLEIK

SEQ ID NO: 20 - protein, 112 aa, Artificial Sequence
  humanized
DVVMTQSPLSLPVTLGQPASISCRSSQSLVHSNGNTYLHWYQQRPGQSPRLLIYKVSNRFSGVPDRFSGSGSGTDFTLKISRVEAEDVGVYYCSQSTHVTWTFGGGTKLEIK

SEQ ID NO: 21 - protein, 8 aa, Mus musculus
GYTFTNYG

SEQ ID NO: 22 - protein, 8 aa, Mus musculus
INTYTGEP

SEQ ID NO: 23 - protein, 13 aa, Mus musculus
ARLGYGKFYVMDY

SEQ ID NO: 24 - protein, 8 aa, Mus musculus
GYTFTSYA

SEQ ID NO: 25 - protein, 8 aa, Mus musculus
GYSFTTYG

SEQ ID NO: 26 - protein, 11 aa, Mus musculus
QSLVHSNGNTY

SEQ ID NO: 27 - protein, 3 aa, Mus musculus
KVS

SEQ ID NO: 28 - protein, 9 aa, Mus musculus
SQSTHVTWT

SEQ ID NO: 29 - protein, 11 aa, Mus musculus
QSLVHSNGNTY